(19) United States
(12) Patent Application Publication
(10) Pub. No.: US 2026/0355122 A1
(43) Pub. Date: Nov. 12, 2026

(54) LIPID NANOPARTICLES FOR INDUCING PROTEIN EXPRESSION IN THE PLACENTA

(71) Applicant: ROWAN UNIVERSITY, Glassboro, NJ (US)

(72) Inventors: Rachel RILEY, Sewell, NJ (US); Samuel Hofbauer, Flanders, NJ (US); Rachel Young, West Berlin, NJ (US)

(21) Appl. No.: 19/707,004

(22) Filed: Jun. 12, 2026

Related U.S. Application Data
(63) Continuation of application No. PCT/US2024/061190, filed on Dec. 19, 2024.
(60) Provisional application No. 63/612,137, filed on Dec. 19, 2023.
(60) Provisional application No. 63/676,802, filed on Jul. 29, 2024.

(51) Int. Cl. A61K 9/51; A61K 9/00; A61K 38/18; A61K 38/20

(52) U.S. Cl. CPC A61K 9/5146; A61K 9/0019; A61K 9/5123; A61K 38/18; A61K 38/2026; A61K 38/2086

(57) ABSTRACT

Disclosed is an ionizable lipid nanoparticle (LNP) composition having four lipid components: C12-200, 1,2-dioleoyl-sn-glycero-3-phosphoethanolamine (DOPE), cholesterol, and (1,2-dimyristoyl-sn-glycero-3-phosphoethanolamine-N-[methoxy(polyethylene glycol)-2000] (ammonium salt)) (DMPE-PEG). The LNP composition is useful for delivering RNA to the placenta, thus also providing methods of treating placenta-related disorders.

### CROSS-REFERENCE TO RELATED APPLICATIONS

This application is a continuation of PCT Application No. PCT/US2024/061190 filed Dec. 19, 2024, which claims priority to and the benefit of U.S. Provisional Patent Application Ser. No. 63/676,802, filed Jul. 29, 2024, and U.S. Provisional Patent Application Ser. No. 63/612,137, filed Dec. 19, 2023, the disclosures of which are hereby incorporated by reference in its entirety.

### STATEMENT REGARDING GOVERNMENT-FUNDED RESEARCH

This invention was made with Federal government support under award numbers 2018266781 and 2301919 by the National Science Foundation Graduate Research Fellowship Program and Engineering Research Initiation, respectively. The Federal government has certain rights in the invention.

This invention was made with an award from the New Jersey Department of Health, under Grant No.: COCR23PRF027, and the New Jersey Health Foundation, under Award PC44-22. This invention was also made with an award from the Peter Joseph Pappas Research Grant.

### SEQUENCE LISTING

This application contains a Sequence Listing that has been submitted electronically as an ST26 xml file named “10859-048US1_Sequence_Listing_2026-06-12.” The xml file, created on Jun. 12, 2026, is 18,700 Bytes in size. The material in the xml file is hereby incorporated by reference in its entirety.

### TECHNICAL FIELD

The present disclosure relates to an ionizable lipid nanoparticle (LNP) composition for delivering RNA to the placenta and methods for treating placenta-related disorders.

### BACKGROUND OF THE INVENTION

The placenta is a vital organ for fetal development, facilitating nutrient and oxygen exchange between the mother and fetus while also serving as a key regulator of immune tolerance at the maternal-fetal interface. Immune regulation plays a critical role in establishing a healthy pregnancy, but the precise biological pathways that govern this process are still under investigation (Deer, E. et al. Nat Rev Nephrol 19, 257-270 (2023); Mor, G., Aldo, P. & Alvero, A. B. Nat Rev Immunol 17, 469-482, (2017). Immune activity at the maternal-fetal interface is a delicate balance between tolerance to the semi-allogeneic fetus and protection against infections, a process that requires intricate control over both pro- and anti-inflammatory signals (Ander, S. E., Diamond, M. S. & Coyne, C. B. Sci Immunol 4, (2019)). Dysregulation of the local immune activity in the placenta and uterine microenvironment is associated with several pregnancy complications, including preeclampsia, intrauterine growth restriction (IUGR), and recurrent pregnancy loss, but the exact mechanisms linking immune dysfunction to these conditions are unclear (Saito, S., Nakashima, A., Shima, T. & Ito, American Journal of Reproductive Immunology 63, 601-610 (2010); Laresgoiti-Servitje, E., Gómez-López, N. & Olson, D. M. Human Reproduction Update 16, 510-524, (2010)).

Lipid nanoparticles (LNPs) have been widely explored for their ability to deliver nucleic acids, such as mRNA, to tissues, and they have shown significant potential in several therapeutic contexts including cancer immunotherapy and vaccines (Riley, R. S., June, C. H., Langer, R. & Mitchell, M. J. Nat Rev Drug Discov 18, 175-196 (2019). However, their application in pregnancy has only recently begun. LNPs may be useful as tools to study how dysregulated immune cell activity, such as macrophages, contribute to healthy and pathologic placenta development. Further, delivering LNPs in early pregnancy may extend the therapeutic window and serve as an immunotherapeutic strategy in high-risk individuals before significant damage occurs.

Macrophages are tissue resident immune cells that have multiple functionalities including phagocytosis of pathogens and dead cells, secretion of cytokines to modulate immune responses, antigen presentation to activate adaptive immunity, and tissue repair and remodeling during wound healing (Shapouri-Moghaddam, A. et al. J Cell Physiol 233, 6425-6440 (2018)). They exhibit a high degree of plasticity to regulate inflammation by adopting either pro-inflammatory or anti-inflammatory phenotypes, depending on the signals they receive from their environment. Due to this plasticity, macrophages are the ideal target for immunomodulatory payloads targeted to a specific tissue (Yao, Y., Xu, X.-H. & Jin, L. Frontiers in Immunology 10, (2019)). Outside of pregnancy, macrophage modulation has gained traction as a therapeutic including cancer, autoimmune disorders, and wound healing, where the ability to polarize macrophages towards pro- or anti-inflammatory states can influence disease progression and tissue repair (Li, J., Jiang, X., Li, H., Gelinsky, M. & Gu, Z. Adv Mater 33, e2004172, (2021)). However, the precise roles of both pro- and anti-inflammatory macrophages during healthy and pathologic pregnancies is in nascent stages. In healthy pregnancies, macrophages support tissue remodeling, placental vascularization, and immune tolerance at the maternal-fetal interface (Yao, Y., Xu, X.-H. & Jin, L. Frontiers in Immunology 10, (2019); Zhao, Q. Y. et al. Front Immunol 13, 994888 (2022)). Conversely, in pregnancy-related disorders, such as preeclampsia, macrophage function favors the pro-inflammatory phenotypes, contributing to poor placental development and adverse pregnancy outcomes (Yao, Y., Xu, X. H. & Jin, L. Front Immunol 10, 792, (2019); Liu, X. et al. Hypertension 79, 2274-2287 (2022)). Given their central role in placental health and disease, a way to modulate macrophage activity to study their role in healthy and diseased pregnancies, and as a potential strategy to address pregnancy complications, is needed.

Placental dysfunction is responsible for severe obstetric complications, such as preeclampsia, Hemolysis, Elevated Liver enzymes, Low Platelet count (HELLP) syndrome, and fetal growth restriction (Burton, G. J., Redman, C. W., Roberts, J. M. & Moffett, BMJ 366, 12381 (2019); Andrikos, A. et al. Arch Gynecol Obstet 305, 597-605 (2022); Barton, J. R. & Sibai, B. M. Clin Perinatol 31, 807-833, vii (2004). The only curative treatment option for some of these, such as severe preeclampsia, is to induce preterm delivery, which may have detrimental impacts on fetal development and survival depending on the stage of gestation (Berzan, E., Doyle, R. & Brown, C. M. Curr Hypertens Rep 16, 473 (2014); Armaly, Z., Jadaon, J. E., Jabbour, A. & Abassi, Z. A. Front Physiol 9, 973 (2018)). Although a complete mechanistic understanding of the pathologies behind preeclampsia and fetal growth restriction remains unknown, several investigations have shown elevated levels of circulating soluble fms-like tyrosine kinase-1 (sFlt-1) and decreased levels of placental growth factor (PIGF) in the blood of pregnant individuals with these conditions (Robinson, C. J., Johnson, D. D., Chang, E. Y., Armstrong, D. M. & Wang, W. Am J Obstet Gynecol 195, 255-259 (2006); Verlohren, S. et al. Am J Obstet Gynecol 206, 58 e51-58 (2012); Verlohren, S. et al. Am J Obstet Gynecol 206, 58 e51-58 (2012); Saffer, C. et al. Pregnancy Hypertens 3, 124-132 (2013); Zeisler, H. et al. N Engl J Med 374, 13-22 (2016)). PIGF contributes to proangiogenic signaling in the placenta by binding vascular endothelial growth factor receptor 1 (VEGFR-1) on endothelial cells (Autiero, M. et al. Nature Medicine 9, 936-943 (2003)). sFlt-1 binds and inactivates PIGF in the circulation, resulting in reduced VEGFR-1 signaling and endothelial dysfunction. A high ratio of sFlt-1:PIGF compared to healthy pregnancy has been proven useful in predicting the development of early-onset preeclampsia and fetal growth restriction (Andrikos, A. et al. Arch Gynecol Obstet 305, 597-605 (2022); Verlohren, S. et al. Am J Obstet Gynecol 206, 58 e51-58 (2012); Zeisler, H. et al. N Engl J Med 374, 13-22 (2016); Gaccioli, F. et al. Hypertension (2022); Agrawal, S., Shinar, S., Cerdeira, A. S., Redman, C. & Vatish, M. Hypertension 74, 1124-1135 (2019); Knudsen, U. B. et al. Pregnancy Hypertens 2, 8-15 (2012)). Extending the duration of pregnancy to a safer time for delivery (ideally >34 weeks) may offer a means to treat placental disorders, improving outcomes for both the fetus and the pregnant person. Thus, there remains a significant need for safe and effective treatment options for placental-related disorders.

### SUMMARY OF THE INVENTION

This disclosure is based, as least in part, on at least the following surprising findings:
(1) a novel ionizable lipid nanoparticle (LNP) composition comprising four lipid components: C12-200 ionizable lipid, 1,2-dioleoyl-sn-glycero-3-phosphoethanolamine (DOPE), cholesterol, and (1,2-dimyristoyl-sn-glycero-3-phosphoethanolamine-N-[methoxy(polyethylene glycol)-2000] (ammonium salt)) (DMPE-PEG).
(2) the LNP composition preferentially accumulates in the placenta and can deliver agents, such as RNA, mRNA, siRNA, etc. to the placenta. Thus, this disclosure provides a novel method of delivery of agents to the placenta and therefore, methods of treating placenta-related disorders.
(3) The LNP composition complexed with placental growth factor (PIGF), Interleukin-4 (IL4) or Interleukin-13 (IL13) mRNA induced protein production and secretion in the placenta. Tissue specific changes in cytokine levels were demonstrated after systemic administration. Thus, this disclosure provides a novel method for vmRNA delivery to the placenta.

The LNP composition includes C12-200 ionizable lipid, 1,2-dioleoyl-sn-glycero-3-phosphoethanolamine (DOPE), cholesterol, and (1,2-dimyristoyl-sn-glycero-3-phosphoethanolamine-N-[methoxy(polyethylene glycol)-2000] (ammonium salt)) (DMPE-PEG). In some embodiments the LNP composition includes about 25% to about 45% C12-200, about 10% to about 22% 1,2-dioleoyl-sn-glycero-3-phosphoethanolamine (DOPE), about 45% to about 55% cholesterol, and about 1.5% to about 3.5% (1,2-dimyristoyl-sn-glycero-3-phosphoethanolamine-N-[methoxy(polyethylene glycol)-2000] (ammonium salt)) (DMPE-PEG). In one embodiment the LNP composition has about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG). In embodiments the LNP composition is complexed with an RNA. The RNA may be mRNA.

In other embodiments, the LNP composition is complexed with placental growth factor (PIGF) mRNA, Interleukin-4 (IL4) mRNA, and/or Interleukin-13 (IL13) mRNA. At least one mRNA may be complexed with one LNP or separate LNPs.

In some embodiments the mRNA:ionizable lipid weight ratio is about 1:2 to about 1:30. In particular embodiments, the mRNA:ionizable lipid weight ratio is about 1:10. LNP sizes range from about 50 nm to about 200 nm.

Additional embodiments relate to methods for treating placenta-related disorders by administering the LNP composition disclosed herein to a pregnant subject suffering from a placenta-related disorder. The administration may be intravenous. Other embodiments may include the LNP composition accumulating in other tissues, such as the spleen or liver.

Another embodiment relates to methods for delivering an mRNA to a placental tissue comprising administering the LNP composition of claim 1 to a pregnant subject, wherein the mRNA is delivered to the placental tissue.

### BRIEF DESCRIPTION OF THE DRAWINGS

FIGS. 1A-C show LNP library formulation and characterization through mixing lipid components (A) using ranges of parameters (B) and assessing in vitro and in vivo (C).

FIGS. 2A-I show characteristics of the LNP library formulations A1-A18: hydrodynamic diameters (A), polydispersity index (B), zeta potential (C), encapsulation efficiency (D), apparent pKa (E), luminescence from BeWos treated with LNPs calculated as a fold change over the PBS-treated group (left axis) and viability as a fold change in absorbance over the PBS-treated group following MTT assay (right axis) (F), luminescence by type of ionizable lipid and phospholipid with each marker representing an individual LNP from the library (G), fold change in luminescence following treatment with LNPs at dosages ranging from 0-100 ng mRNA/well (H), and molar ratios of the five LNPs in the library containing C12-200 and DOPE (I). ***<0.001****<0.0001

FIGS. 3A-E are graphs showing fold change in luminescence plotted against hydrodynamic diameter (A), six LNPs (two center points) formulated with 10, 13, or 16% DOPE and 1.5, 2.0, or 2.5% PEG (B), characterization parameters (C), apparent pKa of the six LNPs and C12-200/DOPE LNPs (D), and apparent pKa of LNPs plotted against the DOPE molar ratio (E). R² coefficient of determination values calculated by linear regression analysis.

FIGS. 4A-C are graphs depicting quantification of normalized radiance with background subtracted for each maternal organ (n=3/treatment group) (A), normalized radiance with background subtracted for all the placentas from each dam 4 hours after treatment with each marker representing the average of all placentas in one dam (B), and liver to placenta (L:P) and spleen to placenta (S:P) delivery ratios for dams treated with LNPs A3, A10, and A14. *p<0.05 (C).

FIGS. 5A-F show LNPs encapsulated with PIGF mRNA as a model for a secreted protein. FIG. 5A shows in vitro expression. PLGF levels in dam serum was collected 24 hours following injection with saline, free mRNA, or LNPs A3, A10, or A14 (n=4) (FIG. 5B). PLGF expression in the (FIG. 5C) dam livers (n=4) and (FIG. 5D) placentas (n=8, 1 placenta from the left and 1 placenta from the right side of the uterine horn per dam). FIG. 5E shows ratio of PLGF expression in the liver compared to the placenta (n=4). *p<0.05**p<0.01***p<0.001.

FIGS. 6A-I show AST and ALT expression in dam serum (A) and fetal liver tissue (B) 24 hours after treatment with LNPs encapsulating PLGF mRNA (n=4), IL-6 concentration in dam serum (C), pg IL-6/mg protein in the placenta (D), ng PIGF/mg protein in the fetal liver (E), the number of fetuses (F), fetal mass (G) placental mass (H) averaged for each dam within each treatment group 24 hours after treatment at the time of extraction (n=4), and ratio of fetal to placental mass averaged for each dam (n=4) (I). *p<0.05**p<0.01

FIG. 7A-E shows a schematic depicting LNP formulation with IL4 mRNA and IL13 mRNA (A), LNP characterization including mean hydrodynamic diameter (B), polydispersity indices (C), and encapsulation efficiencies of LNPs as assessed by Ribogreen assays.

FIGS. 8A-D shows a schematic depicting trophoblast cell lines treated with IL4-LNPs or IL13-LNPs, and the conditioned media (CM) assessed for cytokine content after 12 hours (A). IL4-LNPs and IL13-LNPs elicit dose-dependent ectopic cytokine production in (B) BeWo b30 cells, (C) HTR8/svNEO cells, and (D) JAR cells.

FIGS. 9A-E shows conditioned media (CM) collected from b30 cells treated with IL4-LNPs and IL13-LNPs induces polarization of patient-derived human macrophages by representative flow cytometry data showing the phenotypic shift of patient-derived CD14+CD11c− monocytes after treatment with CM from treated BeWo cells (A). Flow cytometry analysis was used to assess (B) CD209 and (C) HLA-DR expression on CD14+CD11c− cells, and (D) CD206 expression on CD14+CD209+HLA-DR+ cells. CM-treated macrophages were assessed for (E) IL10 secretion by ELISA. *p<0.05 and **p<0.002.

FIGS. 10A and B show decreasing the administered dose of mRNA leads to increased CD206 and CD209 expression. Cells were treated with IL4-LNPs and IL13-LNPs at doses ranging from 0.03-0.5 ng mRNA/uL. Flow cytometry was used to assess (A) CD206 expression on CD14+CD209+HLA-DR+ cells, and (B) CD209 expression on and CD14+CD11c− cells. *p<0.05.

FIGS. 11A-D are graphs showing quantification of the average radiance from IVIS imaging. Each marker represents a different dam. IL4 and IL13 levels in the placenta (A), liver (B), and (C) spleen 4 hours after LNP injections (0.5 mg mRNA/kg). *p<0.05, **p<0.01, and ****p<0.0001.

FIGS. 12A-D are graphs showing cytokine levels in dam serum following LNP injections: Luminex assay quantification of cytokine levels in dam serum 4 hours (A) and 48 hours (B) after injections of PBS, Luc-LNPs, or IL4-LNPs+IL13-LNPs; IL4 (left) and IL13 (right) levels in the serum 4 hours (C) and 48 hours (D) after injections. (*p<0.05, **p<0.01, ***p<0.001 and ****p<0.0001)

### DESCRIPTION

The present disclosure relates to a composition including a lipid nanoparticle (LNP) and method for effective delivery of ribonucleic acid (RNA) molecules. The disclosed compositions and methods are useful for development of and/or as therapeutics to treat disorders in organs or tissues where the LNP preferentially accumulates.

Embodiments include an ionizable lipid nanoparticle (LNP) composition (herein “LNP,” “composition,” and/or “LNP composition” interchangeably) comprising four lipid components: C12-200, 1,2-dioleoyl-sn-glycero-3-phosphoethanolamine (DOPE), cholesterol, and (1,2-dimyristoyl-sn-glycero-3-phosphoethanolamine-N-[methoxy(polyethylene glycol)-2000] (ammonium salt)) (DMPE-PEG). In an embodiment, the LNP composition includes about 25% to about 45% C12-200, about 10% to about 22% 1,2-dioleoyl-sn-glycero-3-phosphoethanolamine (DOPE), about 45% to about 55% cholesterol, and about 1.5% to about 3.5% (1,2-dimyristoyl-sn-glycero-3-phosphoethanolamine-N-[methoxy(polyethylene glycol)-2000] (ammonium salt)) (DMPE-PEG). In another embodiment the LNP composition has about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG). In one embodiment the LNP composition comprises about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG). In another embodiment the LNP comprises about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG).

In embodiments, the LNP composition preferentially accumulates in specific organs or tissues, for example and without limitation, the placenta. In other embodiments, the LNP composition preferentially accumulates in the spleen and/or liver. In some embodiments, the LNP may include a targeting ligand to further increase specific accumulation.

In some embodiments the disclosure relates to methods for delivering agents, such as RNA, to specific organs and tissues, such as the placenta for example and without limitation.

In other embodiment the lipid components or LNPs are complexed with nucleic acids, such as and without limitation RNA, mRNA, siRNA, microRNA, DNA, etc., also sometimes described as encapsulating the nucleic acids. In one embodiment, the LNP is complexed with an Interleukin-4 (IL4) nucleic acid molecule, for example the nucleic acid sequence as set forth in SEQ ID NO. 3 or mRNA derived from SEQ ID NO. 3, or an Interleukin-13 (IL13) nucleic acid molecule, for example the nucleic acid sequence as set forth in SEQ ID NO. 2 or mRNA derived from SEQ ID NO. 2, enabling protein production and secretion in the placenta, for example. In another embodiment, the LNP is complexed with a placental growth factor (PIGF) nucleic acid molecule, for example nucleic acid sequence as set forth in SEQ ID NO. 1 or mRNA derived from SEQ ID NO. 1.

In other embodiments methods of treating placental-related disorders are disclosed. In an example embodiment, the disclosure relates to treating preeclampsia. In other embodiments the disclosure relates to treatment of fetal growth restriction or other applications requiring gene therapy.

In embodiments patients are treated by administering the LNP, which may be complexed with a specific nucleic acid, e.g. mRNA. In an example embodiment, administration is by injection, such as intravenous. “Patient,” “subject,” or “individual” refers to an animal, including a mammal, preferably a human, a monkey, a chimpanzee, a horse, a cow, a sheep, a goat, a pig, a cat, a dog, a mouse, a rat, or a rabbit.

Additional embodiments include using the LNP compositions as research tools, such as in methods of studying accumulation in specific areas, such as organs, by encapsulating luciferase mRNA, for example. In other non-limiting examples, the LNPs may be used to study placenta development or pathophysiology of pregnancy-related diseases.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not intended to limit the scope of the invention.

### Example 1

Formulation of LNPs: C12-200 and DLin-MC3-DMA (MC3) was purchased from MedChem Express (Monmouth Junction, NJ). Other LNP components including cholesterol, DSPC, DOPE, and DMPE-PEG2000 (ammonium salt)) were purchased from Avanti Polar Lipids Inc. (Birmingham, AL). Codon optimized mRNA, SEQ ID NO. 1, was prepared by in vitro transcription through a collaboration with the Engineered mRNA and Targeted Nanomedicine core facility at the University of Pennsylvania (Philadelphia, PA). Firefly luciferase and PIGF mRNA (transcript variant 1, NM_002632.6) were co-synthesized with 1-methylpseudouridine modifications, and co-transcriptionally capped using the CleanCap system (TriLink) and purified using cellulose based chromatography (PMID: 30933724).

Each LNP was formulated via mixing with micropipettes by combining one volume of lipid/ethanol mixture to three volumes of mRNA in citrate buffer (1:3 ethanol:citrate volume ratio). The lipid mixture for each LNP formulation contained various molar ratios of ionizable lipid:phospholipid:cholesterol:PEG. mRNA was diluted in citrate buffer (pH 3) to an mRNA:ionizable lipid weight ratio of 1:10 for all LNP formulations. After mixing, the LNPs were dialyzed against PBS (pH 7.4) for 2 hours, sterile filtered using 0.2 μm filters, and stored at 4 °C.

Characterization of LNPs: The LNPs in Library A were formulated a total of three separate times to measure consistency in LNP characterization parameters and to complete all experiments. Dynamic light scattering (DLS) measurements and mRNA encapsulation efficiency, as described below, were measured for each LNP formulation in each batch of the library. The average and standard deviation of the hydrodynamic diameter, polydispersity index, and encapsulation efficiency of each LNP is reported for all three batches of the library. Each LNP formulation was diluted 1:100 in deionized water in cuvettes and DLS measurements were run on a Malvern Zetasizer Nano ZS (Malvern Panalytical, Malvern, UK). Samples were diluted 1:100 in deionized water in folded capillary cuvettes and zeta potential was measured in triplicate on the Zetasizer Nano ZS with an applied voltage of 150 volts.

The encapsulation efficiency of each LNP formulation was calculated using QuantiFluor® RNA System (Promega, Madison, WI) as previously described (Riley, R. S. et al. (2021), Science Advances 7:1-15). Briefly, LNPs were diluted 1:100 in 1×TE buffer in two microcentrifuge tubes per LNP formulation. 1% v/v Triton X-100 (Thermo Scientific, Waltham, MA) was added to one of the tubes and both were heated to 37 °C and shaken at 600 RPM for 5 mins, followed by cooling to room temperature for 10 mins. LNP samples and RNA standards were plated in triplicate in black 96-well plates and the fluorescent reagent was added per the manufacturer instructions. Fluorescent intensity was read on the plate reader (excitation, 492 nm; emission, 540 nm). Background signal was subtracted from each well and triplicate wells for each LNP were averaged. RNA content was quantified by comparing samples to the standard curve, and encapsulation efficiency (%) was calculated according to the equation

$$EE = \frac{B - A}{B} \times 100 ,$$

where A is the RNA content in samples without Triton X-100 treatment (intact LNPs) and B is the RNA content in samples treated with Triton X-100 (lysed LNPs).

The apparent pKa of LNPs was determined via TNS [6-(p-toluidinyl) naphthalene-2-sulfonic acid] (Thomas Scientific, Swedesboro, NJ) assays (Heyes, J., et al. (2005), J. Control Release, 107:276-287). Briefly, a buffer solution of 150 mM sodium chloride, 20 mM sodium phosphate, 20 mM ammonium acetate, and 25 mM ammonium citrate (VWR Chemicals BDH, Radnor, PA) was separated into 21 varied pH solutions, adjusted from pH 2 to 12 in increments of 0.5 pH. 2.5 μL of each LNP formulation was combined with 125 μL of each pH-adjusted solution in black 96-well plates in triplicate. TNS was added to each well for a final TNS concentration of 6 μM and the fluorescence intensity was read on a plate reader (Molecular Devices, San Jose, CA) (excitation, 322 nm; emission, 431 nm). Fluorescence intensity versus pH was plotted, and apparent pKa was calculated as the pH corresponding to 50% of its maximum value, representing 50% protonation.

In vitro transfection of LNPs with luciferase or PIGF mRNA:LNPs in in the library (A1-A18) were formulated with luciferase mRNA as a reporter molecule and a luciferase assay was performed to measure transfection and mRNA translation in cells. The b30 clone (Wice, B. et al. (1990), Experimental Cell Research, 186:306-316) of the BeWo choriocarcinoma cell line (termed “BeWos” herein) were cultured in F-12K Nutrient Mixture (Kaighn's Mod.) with L-glutamine (Corning Inc., Corning, NY) supplemented with 10% fetal bovine serum (Avantor, Radnor Township, PA) and 1% penicillin/streptomycin (VWR, Radnor, PA). Cultures were grown in an incubator set at 37 °C with 5% CO₂. Cells were plated at 20,000 cells per well in 96-well plates with 200 μL of complete culture media in triplicate for each LNP formulation. After 4 hours, cells were treated with LNPs diluted in sterile PBS at 20-100 ng mRNA/well or sterile PBS as the negative control. Luciferase expression was analyzed after 24 hours per manufacturer instructions (Promega, Madison, WI). Cells were washed with sterile PBS and 20 μL of 1× lysis buffer was added to each well. After 10 minutes of incubation at room temperature, cells were centrifuged at 12,000×g for 2 minutes, and lysates were plated into white 96-well plates. 100 μL of luciferase assay substrate was added to each well and the luminescent signal was quantified using the plate reader. The average luminescent signal from each group was normalized to untreated cells and reported as the fold change in luminescence. Statistical analysis of luciferase expression from the LNP library screen was conducted (see “Statistical Analysis” section below).

BeWos were treated with LNPs formulated with PIGF mRNA and free mRNA as described above. After 24 hours of incubation with LNPs, cell culture supernatant was collected and centrifuged at 2,000×g and 4 °C for 5 minutes to remove cell debris. The supernatant was assayed for PIGF concentration using an enzyme-linked immunosorbent assay (ELISA) per manufacturer instructions (Rockland Immunochemicals, Inc, Pottstown, PA). Briefly, biotinylated anti-human PIGF antibody was used to measure PIGF content in samples via a reaction of avidin-biotin peroxidase complex and 3,3′5,5′-tetramethylbenzidine (TMB) substrate. After color development, stop solution was added to the assay plate and absorbance was read at 450 nm on a microplate reader. Sample absorbance values were compared to a standard curve to calculate PIGF concentration.

LNP Toxicity Analysis: To assess metabolic activity as an indicator of cell viability, BeWos were plated as described above and treated with 100 ng mRNA/well of each LNP formulation. After 24 hours, cells were assayed using the MTT (3-(4,5-dimethylthiazol-2-yl)-2,5-diphenyltetrazolium bromide) tetrazolium reduction assay (BioVision, Milpitas, CA) according to manufacturer instructions. Briefly, cells were washed with sterile PBS and 50 μL of serum-free culture media and 50 μL of MTT reagent were added to each well. After incubation for 3 hours at 37 °C, 150 μL of MTT solvent was added to each well. The plate was rocked for 15 mins at room temperature in the dark and absorbance at 590 nm was read on the plate reader. The average absorbance of wells containing no cells was subtracted as background from each well. The absorbance signal from each group was normalized to untreated cells and reported as the fold change in absorbance.

Administration and Biodistribution of LNPs In Vivo: Female mice between 8-39 weeks (mean 22.0 weeks) of age were maintained, bred, and used in accordance with Animal Use Protocols approved by the Institutional Animal Care and Use Committee at the University of Delaware (AUP #1320 and #1341). Timed-pregnant CD1 mice were bred and separated 12 hours later, denoted as E0.5. At E17.5, dams were injected intravenously via the tail vein with 0.5 mg mRNA/kg mouse of LNPs A3, A10, or A14, or the equivalent volume of saline (n=3 dams per treatment group). After 4 hours, dams were injected intraperitoneally with d-luciferin with potassium salt (150 mg/kg) (Biotium, Fremont, CA). Anesthetized dams were placed supine into the IVIS Lumina III (PerkinElmer, Waltham, MA), and the luminescence signal was detected. Dams were then sacrificed, and the blood was collected via cardiac puncture with a 25-gauge needle and syringe prefilled with 100 μL of 0.5 M EDTA (pH 8). Blood was centrifuged at 2,000×g at 4 °C for 10 minutes to separate, and the top plasma layer was transferred into a clean tube and stored at −80 °C. Maternal organs (liver, spleen, pancreas, kidneys, ovaries, heart, and lungs), placentas, and fetuses were excised and imaged separately by IVIS. The weights of all placentas and fetuses were measured via mass balance. Following imaging, maternal organs and placentas were immediately placed on dry ice and stored at −80 °C. Fetal livers were excised from 5 fetuses per dam and immediately placed on dry ice and stored at −80 °C.

Image analysis was conducted in the Living Image software (PerkinElmer, Waltham, MA). To quantify luminescent flux, an ROI was placed over each placenta or dam organ of interest. The average radiance [p/s/cm2/sr] of the ROI with background subtracted for all placentas within each dam were averaged. Next, the average of all of the placentas per replicate dam (n=3) was calculated. Similarly, the average radiance of the ROI with background subtracted for each dam organ was averaged for the replicate mice (n=3) treated with each LNP formulation. The liver:placenta and spleen:placenta delivery ratios for each LNP formulation were calculated by dividing the average liver or spleen radiance by the average placental radiance per replicate dam (n=3), shown with the standard error of the mean.

LNP-Mediated Delivery of PIGF mRNA: Dams (9-23 weeks (mean 13.0 weeks)) at E17.5 were injected via the tail vein with 0.5 mg mRNA/kg mouse weight with free PIGF mRNA, LNPs A3, A10, A14, or the equivalent volume of saline (n=4 dams per treatment group). Four hours after injection, blood was collected via the submandibular vein of the dam with a 25-gauge needle. Twenty-four hours after injection, dams were sacrificed, and blood was collected via cardiac puncture with a 25-gauge needle and with a syringe prefilled with 100 μL of 0.5 M EDTA (pH 8). Both blood samples were immediately centrifuged after collection at 2,000×g at 4 °C for 10 minutes. The top plasma layer was transferred into a clean tube and stored at −80 °C. Placentas and fetuses were excised, rinsed in PBS, and measured using a mass balance. Following measurement, placentas were placed on dry ice and stored at −80 °C. Fetal livers were excised from 5 fetuses per dam and immediately placed on dry ice and stored at −80 °C. Maternal organs (liver, spleen, kidneys, ovaries, lungs, and uterine horn) were surgically excised and immediately placed on dry ice and stored at −80 °C.

The liver and two placentas from each dam were digested to extract protein for PIGF analysis by ELISA. Frozen tissue samples were digested with 300 μL of M-PER digestion reagent (Pierce Biotechnology, Rockford, IL) supplemented with 1× protease and phosphatase inhibitor cocktail (Pierce Biotechnology, Rockford, IL) per 5 mg of tissue on ice. Mechanical grinding of tissues was performed with disposable tissue grinders (Kimble Chase Life Science, Rockwood, TN) per manufacturer instructions. Tissue lysates were kept on ice for one hour with intermittent 30-seconds of vortexing and sonication every 15 minutes. RBC lysis buffer (BioLegend, San Diego, CA) was added to 1× in the lysate solution incubated on ice for 10 mins. Lysates were centrifuged at 12,000×g for 10 min (4 °C) and the supernatant was transferred to a new tube and stored at −80 °C until analysis. Prior to analysis, lysates were thawed on ice and centrifuged at 12,000×g for 10 min (4 °C) to remove debris. Liver, plasma, and placentas were assayed for PIGF concentration using an ELISA per manufacturer instructions, as described above (Rockland Immunochemicals, Inc, Pottstown, PA).

Toxicity Analysis: Liver enzymes ALT and AST were measured using colorimetric assay kits (Cayman Chemical, Ann Arbor, MI) per manufacturer instructions. Briefly, samples and controls were added to the assay plate with substrate and cofactor and incubated at 37 °C for 15 minutes. Initiator was added to the assay plate and absorbance immediately measured at 340 nm once every minute for 10 minutes at 37 °C on a microplate reader. The absorbance values were plotted as a function of time and slope was found for the linear portion of the curve. Activity was calculated according to the equation Activity

$$( \frac{U}{mL} ) = \frac{\frac{Δ {A}_{340}}{min} \times 0.21 mL}{4.11 {mM}^{- 1} \times 0.02 mL}$$

where activity is ALT or AST activity. ALT and AST assays were performed on fetal liver tissue lysates and in the dam plasma (both prepared as described above). Dam serum and placental tissue lysates (both prepared as described above) were assayed for IL-6 concentration using an ELISA per manufacturer instructions (Invitrogen, Waltham, MA). Sample absorbance values were compared to a standard curve to calculate IL-6 concentration.

Statistical Analysis: Analysis of the DSD was conducted in JMP Pro 16 (SAS Institute Inc., Cary, NC) software using the fit definitive screening platform, while all other analyses were performed using GraphPad Prism 9.0 (San Diego, CA). JMP Pro 16 uses effective model selection for DSDs to identify design variables as active main or pairwise interactions when the p-value computed using the t Ratio and degrees of freedom for error is less than 0.05 (Jones, B., Nachtsheim, C. J. (2017), Technometrics 59:319-329). After active effects are identified in the Combined Model Parameter Estimates report, a standard least squares fit is applied to obtain the significant effects in the fit model.

All experiments have n=3 replicates unless otherwise indicated. Continuous features were assessed for normality using D'Agostino-Pearson omnibus (K2), Anderson-Darling (A2*), Shapiro-Wilk (W), and/or Kolmogorov-Smirnov (distance). Luciferase expression across the different LNPs in Library A, PIGF content in the dam serum and placentas, AST content in the dam serum, number of fetuses and the weights of the fetuses between treatment groups in the in vivo study were non-normal. Thus, all were analyzed via the Kruskal-Wallis test followed by pairwise comparisons of the different types of LNPs and/or treatment groups using Dunn's method for multiplicity adjustment. An ordinary one-way ANOVA was used to compare the normally distributed PIGF content in the dam livers, dam ALT and fetal ALT and AST levels, IL-6 content in dam serum and placentas, and the weights of the placentas between treatment groups in the in vivo study followed by pairwise comparisons of different types of LNPs adjusted for multiplicity using Tukey's method. Results are represented as mean with standard error of the mean (SEM) and statistical significance was determined at 0.05 (*), 0.01 (**), 0.001 (***), or 0.0001 (****).

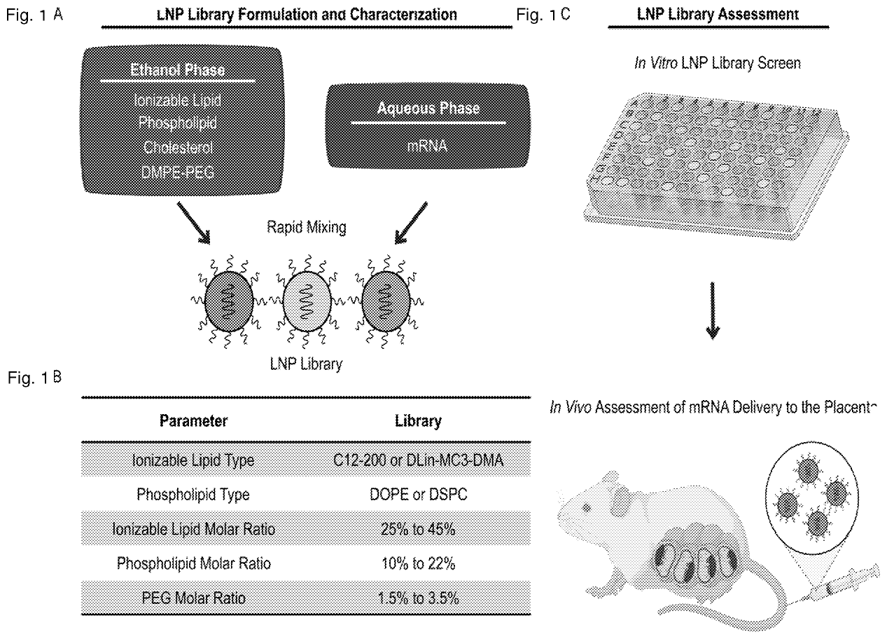

LNP Library Formulation and Characterization. A definitive screening design (DSD) was used to create a library of 18 chemically unique LNPs (A1-A18) from the design space available, as previously described (Kauffman, K. J. et al. (2015), Nano Lett 15:7300-7306; Jones B. and Nachtsheim, C. J. (2013), J. Quality Technology 45; Jones B. and Nachtsheim, C. J. (2011), J. Quality Technology 43; Jones B. and Nachtsheim, C. J. (2017), Technometrics 59:319-329). A DSD is a DOE approach commonly used for early-stage experimentation involving a combination of three-level continuous and two-level categorical factors to identify linear and quadratic effects. LNP formulation parameters were used as factors in the DSD. Two categorical factors were used-type of ionizable lipid and type of phospholipid- and three continuous factors were used-molar percentages of ionizable lipid, phospholipid, and (1,2-dimyristoyl-sn-glycero-3-phosphoethanolamine-N-[methoxy(polyethylene glycol)-2000] (ammonium salt)) (DMPE-PEG). Two established ionizable lipids were used, C12-200 or DLin-MC3-DMA, to assess how ionizable lipid structure impacts delivery to trophoblasts. C12-200 has been evaluated in LNPs for both siRNA and mRNA delivery in a variety of cell types and animal models (Kauffman, K. J. et al. Nano Lett 15, 7300-7306 (2015); Dahlman, J. E. et al. Proc Natl Acad Sci USA 114, 2060-2065 (2017); Hajj, K. A. et al. Small 15, e1805097 (2019); Love, K. T. et al. Proc Natl Acad Sci USA 107, 1864-1869 (2010); Oberli, M. A. et al. Nano Lett 17, 1326-1335 (2017); Whitehead, K. A. et al. Nat Commun 5, 4277 (2014)). DLin-MC3-DMA is the ionizable lipid in the FDA-approved therapy to treat hereditary transthyretin-mediated amyloidosis (Adams, D. et al. N Engl J Med 379, 11-21 (2018); Keam, S. J. Drugs 82, 1419-1425 (2022)). LNP delivery was compared using two phospholipids, 1,2 distearoyl-sn-glycero-3-phosphocholine (DSPC) or 1,2-dioleoyl-sn-glycero-3-phosphoethanolamine (DOPE) (FIG. 1A). The molar percentages of ionizable lipid (25-45%), phospholipid (10-22%), and DMPE-PEG (1.5-3.5%) used was varied to make LNPs (Kauffman, K. J. et al. Nano Lett, 15:7300-7306 (2015); Whitehead, K. A. et al. Nat Commun 5, 4277 (2014)) (FIG. 1B). The remaining molar composition (to add up to 100%) for each LNP was cholesterol. This design choice was based on prior literature showing that the types and amounts of ionizable lipid, phospholipid, and PEG influence hepatocellular mRNA delivery (Akinc, A. et al. Nat Biotechnol 26, 561-569 (2008); Kauffman, K. J. et al. Nano Lett 15, 7300-7306 (2015); Jayaraman, M. et al. Angew Chem Int Ed Engl 51, 8529-8533 (2012); Hajj, K. A. et al. Small 15, e1805097 (2019); Love, K. T. et al. Lipid-like materials for low-dose, in vivo gene silencing. Proc Natl Acad Sci USA 107, 1864-1869 (2010)). Thus, these as factors were included in the DSD to investigate their effect on trophoblast mRNA delivery, and the amount of cholesterol in each formulation was calculated as the remainder of each recipe. Since the DSD did not include cholesterol as an independent factor, it could not be included in the statistical analysis. In initial studies, luciferase mRNA was encapsulated into LNPs as it is detectable and quantifiable using a plate reader for in vitro experiments and via an In Vivo Imaging System (IVIS) for in vivo studies.

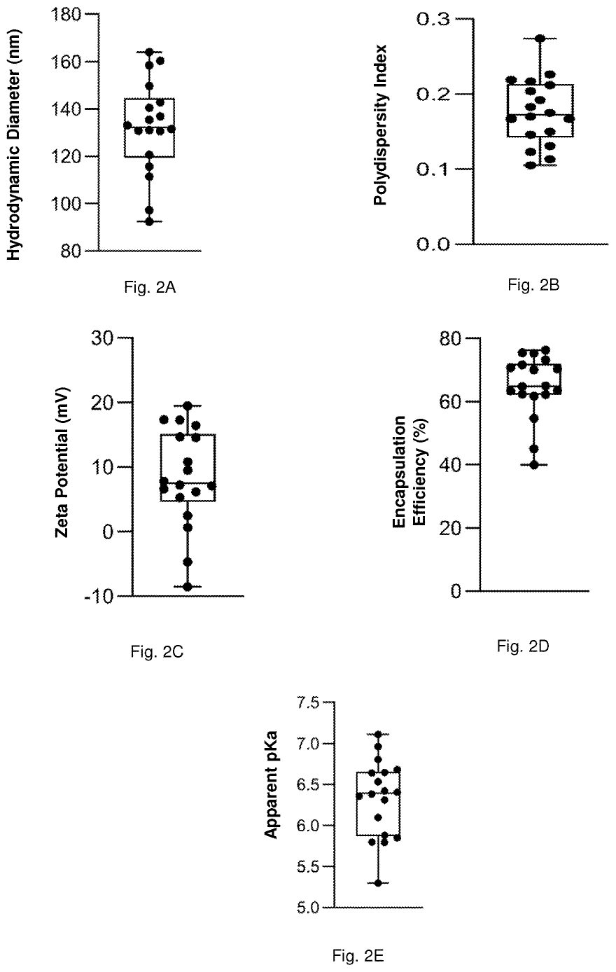

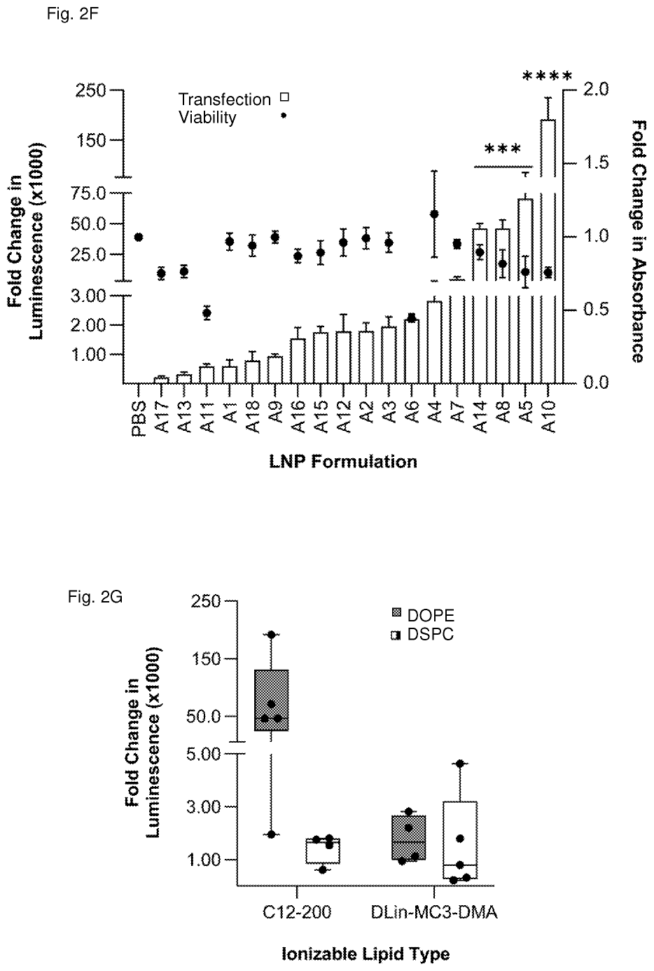

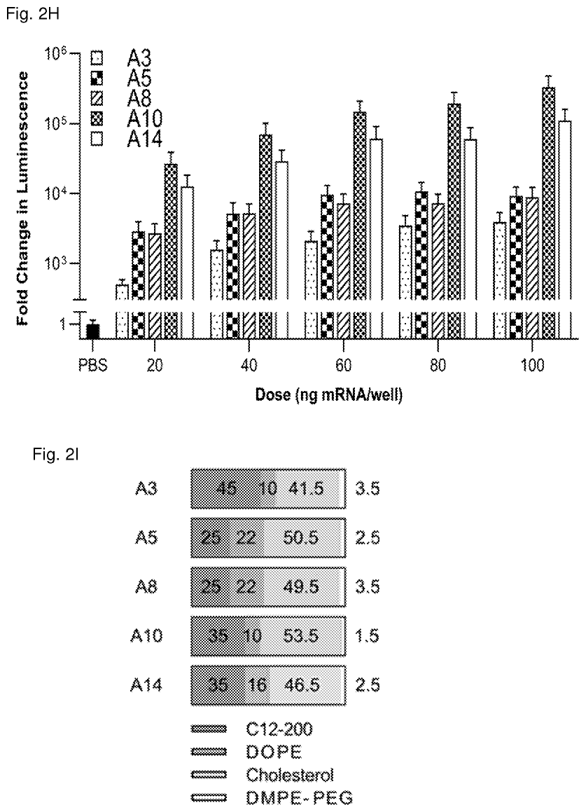

The hydrodynamic diameters of LNPs in the library ranged from 92.4 to 164.0 nm (FIG. 2A) and the polydispersity indices (PDI) ranged from 0.120 to 0.317 (FIG. 2B). The zeta potential of LNPs in the library ranged from −8.52 to 19.45 mV (FIG. 2C). To track LNP stability over time, the hydrodynamic diameter and PDI of LNPs was measured 50 and 100 days following formulation. LNPs were stored at 4 °C for the duration of the experiment. The PDI of some LNPs increased over time while in storage, ranging from 0.170 to 0.586. This may indicate a less monodisperse solution over the 100 day test period, which could result from LNP aggregation or degradation (Danaei, M. et al. Pharmaceutics 10 (2018)). The mRNA encapsulation efficiency was also characterized, which ranged from 35.6% to 83.2% encapsulation relative to the amount of mRNA added during formulation (FIG. 2D). To evaluate encapsulation stability over time, the encapsulation efficiency was calculated one month post-formulation for select LNPs. The encapsulation efficiencies for LNPs A5, A8, A10, and A14 decreased <20% over one month, while the encapsulation efficiency for LNP A3 decreased 28%. Together, these results indicate that encapsulation is stable over one month, but future studies would need to be conducted to evaluate longer timepoints.

The surface ionization was evaluated by a 6-(p-toluidinyl) naphthalene-2-sulfonic acid (TNS) assay and reported as the apparent pKa (FIG. 2E), ranging from 5.298 to 7.111. Apparent pKa measured in this way represents the pH at which half of the ionizable lipids are protonated to induce endosomal escape and cytoplasmic mRNA delivery (Hajj, K. A. et al. Small 15, e1805097 (2019); Whitehead, K. A. et al. Degradable lipid nanoparticles with predictable in vivo siRNA delivery activity. Nat Commun 5, 4277 (2014)). A DSD fit analysis was utilized to identify which LNP formulation parameters, defined as factors in the DSD, influence apparent pKa as main effects or pairwise interactions. Through this analysis, a main effect is defined as the effect of a single LNP formulation parameter on the apparent pKa, and a pairwise interaction is the combined effect of two LNP formulation parameters on the apparent pKa. The type of ionizable lipid was a main effect for apparent pKa (p<0.001) with C12-200 in LNPs yielding lower apparent pKa values compared to DLin-MC3-DMA. The 18 LNPs formulated through our DSD were used to assess luciferase mRNA delivery in vitro and in vivo, as described below.

LNP Composition Dictates Delivery to Trophoblasts. To assess how LNP composition impacts delivery in vitro, BeWo b30 trophoblast cells (referred to as BeWos hereafter) were treated with each LNP at 0 or 100 ng/well for 24 hours. LNP A10 yielded ~190,000-fold higher luciferase expression compared to phosphate-buffered saline (PBS)-treated cells (p<0.0001, FIG. 2F). LNPs A5, A8, and A14 had the next highest luciferase expression compared to PBS-treated cells (p<0.001, FIG. 2F). Interestingly, these four top-performing LNPs were comprised of C12-200 and DOPE as the ionizable lipid and phospholipid, respectively (FIG. 2G). No LNPs prepared with DLin-MC3-DMA or DSPC yielded high luciferase expression (FIG. 2G), beyond 45,000-fold above the PBS-treated cells. This importance in ionizable lipid choice aligns with prior work that demonstrated that the ionizable lipid structure, and in particular the polyamine headgroup, drives delivery efficiency (Chaudhary, N. et al. bioRxiv, 2023.2002.2015.528720 (2023). Based on our results demonstrating LNPs with C12-200 and DOPE yielded the highest mRNA delivery (FIG. 2G), all five LNPs from Library A containing both C12-200 and DOPE-LNPs A3, A5, A8, A10, and A14 were assessed. Following a dose-response experiment in BeWos, these five LNPs showed large differences in luminescence despite containing the same lipids (FIG. 2H). Similar to the initial screen, LNP A10 and LNP A3 had the highest and lowest expression of these five LNPs, respectively, at all doses (FIG. 2H). In addition to luciferase expression, BeWo viability was assessed following LNP treatment using MTT (3-(4,5-dimethylthiazol-2-yl)-2,5-diphenyltetrazolium bromide) tetrazolium reduction assays (referred to as MTT hereafter). Only cells treated with LNPs A6 (p=0.011) and A11 (p=0.023) had reduced viability compared to the PBS-treated cells (FIG. 2F), indicating that the majority of LNP formulations are not toxic to BeWos.

The DSD was analyzed to determine the important LNP parameters affecting viability of BeWos. This demonstrated that the type of ionizable lipid (p=0.005), type of phospholipid (p=0.018), ionizable lipid amount (p<0.0001) and phospholipid amount (p=0.016) were significant factor. The two LNPs that resulted in significantly lower viability compared to the controls were comprised of low amounts of DLin-MC3-DMA and DOPE-25% and 10%, respectively-which was confirmed by our DSD analysis. Additionally, the model found pairwise interactions between type of phospholipid and phospholipid amount (p<0.0001), and type of phospholipid and ionizable lipid amount (p<0.0001).

Next, the DSD was evaluated for factors affecting transfection. The type of ionizable lipid (p=0.018) and type of phospholipid (p=0.017) were significant factors affecting transfection, with C12-200 or DOPE in LNPs yielding the strongest luciferase expression overall compared to the other LNP lipid components. Additionally, the model found several pairwise interactions between the type of ionizable lipid and PEG amount (p=0.036), type of phospholipid and PEG amount (p=0.034), and type of ionizable lipid and type of phospholipid (p=0.0105). This indicates that the mechanism by which each LNP parameter affects LNP transfection is more complicated than an additive manner of main effects, as it involves several pairwise interactions. Based on this, it is pertinent to study both the main effects and pairwise interactions when researchers are developing LNPs for nucleic acid delivery. In particular, the pairwise interactions revealed that maximal delivery occurs when C12-200 and DOPE are both included in the LNP formulation (p=0.0105). This finding agrees with prior literature comparing mRNA delivery with LNPs made with DOPE or DSPC, as the use of DOPE was found to yield higher transfection than LNPs made with DSPC (Kauffman, K. J. et al. Nano Lett 15, 7300-7306 (2015), Oberli, M. A. et al. Nano Lett 17, 1326-1335 (2017)). A limitation of DSDs to build a LNP library is the possibility of other high-performing LNPs potentially missed within the design space. Although it is not feasible to test every LNP formulation (as there are thousands of possible combinations), our data shows that C12-200 and DOPE are the key drivers for mRNA delivery to trophoblasts.

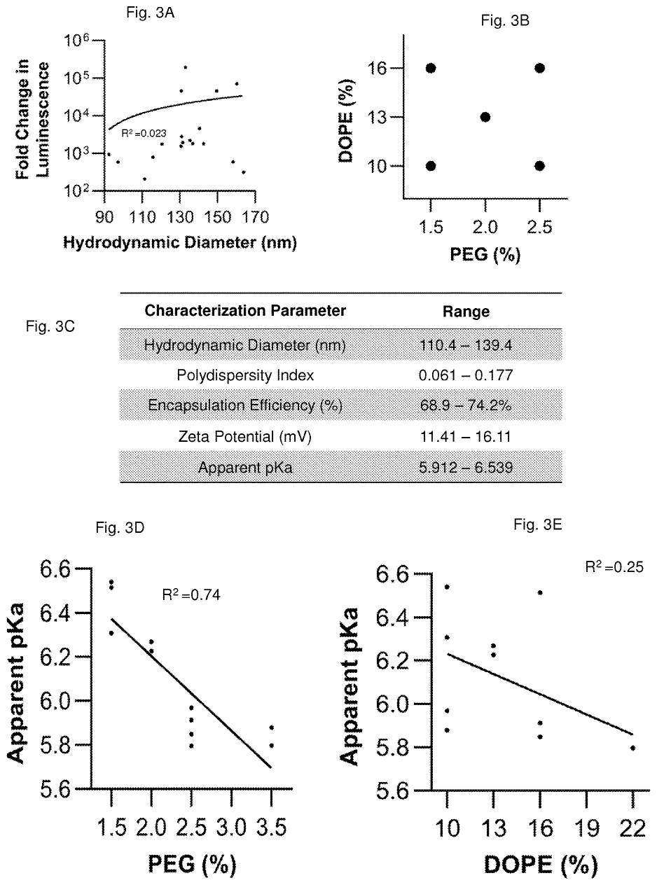

To consider the influence of hydrodynamic diameter on mRNA delivery, the hydrodynamic diameter versus luciferase expression was fit with a linear regression, which had an R² coefficient of determination of 0.023 (FIG. 3A). This indicates that there is not a linear relationship between hydrodynamic diameter and luciferase expression, suggesting that LNP size does not significantly influence LNP delivery. To further confirm that size does not drive delivery, LNPs were grouped into three size categories-<120 nm, 130-139 nm, >140 nm—and compared the delivery efficiency, finding no significant difference in delivery. Therefore, variance in luciferase expression may be due to differences in the molar ratios of each lipid component within the LNPs affecting the apparent pKa (Shobaki, N., Sato, Y. & Harashima, H. Int J Nanomedicine 13, 8395-8410 (2018)). For example, LNP A10 is comprised of C12-200:DOPE:Cholesterol:PEG molar ratios of 35:10:53.5:1.5 and has an apparent pKa of 6.308; by comparison, LNP A14 is comprised of the molar ratios 35:16:46.5:2.5 and has an apparent pKa of 5.848 (FIG. 2I). To explore which LNP component is driving the differences in delivery, the apparent pKa was plotted versus the PEG and DOPE molar ratio for LNPs A3, A5, A8, A10, and A14 and observed that LNPs containing a lower PEG molar ratio had a higher apparent pKa. Comparatively, there was no relationship between DOPE, cholesterol, or C12-200 molar ratio and apparent pKa.

To further investigate how PEG and DOPE molar ratios impact delivery, three new LNPs in addition to the original A10 and A14 formulations were prepared. These new formulations contained the lower, center, and higher molar ratios of PEG and DOPE from LNPs A10 and A14 (FIG. 3B). The differences in PEG and DOPE molar ratios were compared because A10 and A14 have similar formulation parameters-both contain 35% C12-200—but show large differences in luciferase expression in vitro (FIG. 2F, H). The hydrodynamic diameter, polydispersity index, mRNA encapsulation efficiency, zeta potential, and apparent pKa were measured for these new LNPs (FIG. 3C). The apparent pKa of the new LNPs and Library A LNPs that contained C12-200 and DOPE (LNPs A3, A5, A8, A10, and A14) were compared versus the PEG (FIG. 3D) and DOPE molar ratios (FIG. 3E). The data was fit with a linear regression with R² coefficients of determination of 0.736 for apparent pKa versus PEG and 0.247 for apparent pKa versus DOPE (FIG. 3D-E). The linear relationship between apparent pKa and PEG molar ratio indicates that a lower PEG molar ratio results in a higher apparent pKa. Here, a PEG molar ratio of 1.5% resulted in an apparent pKa between 6.3 and 6.5. The LNP with the highest mRNA delivery in BeWos, LNP A10, contained 1.5% PEG and had an apparent pKa of 6.308. The pKa of LNP A10 aligns well with previous studies demonstrating that pKa values between 6.0-6.5 may be ideal for in vivo siRNA delivery (Jayaraman, M. et al. Angew Chem Int Ed Engl 51, 8529-8533 (2012), Hajj, K. A. et al. Small 15, e1805097 (2019), Whitehead, K. A. et al. Nat Commun 5, 4277 (2014)). LNPs A3, A5, A8, and A14 had higher PEG molar ratios of 3.5%, 2.5%, 3.5%, and 2.5% and lower apparent pKa's of 5.878, 5.795, 5.798, and 5.848, respectively. The lower apparent pKa values of these LNPs, compared to A10, indicate a less protonated LNP surface, which can decrease cellular uptake and endosomal escape, as demonstrated by the in vitro data (FIG. 2F, H) (Carrasco, M. J. et al. Commun Biol 4, 956 (2021)).

Based on the in vitro delivery results and the low toxicity of these LNPs, LNPs A3, A14, and A10 were selected as low, medium, and high-performing LNPs for further studies. Importantly, LNPs A3, A14, and A10 also exhibited similar encapsulation efficiencies (61.75-63.35%) and hydrodynamic diameters (130.8-133.0 nm), allowing direct comparison to their delivery efficiency based on their lipid compositions.

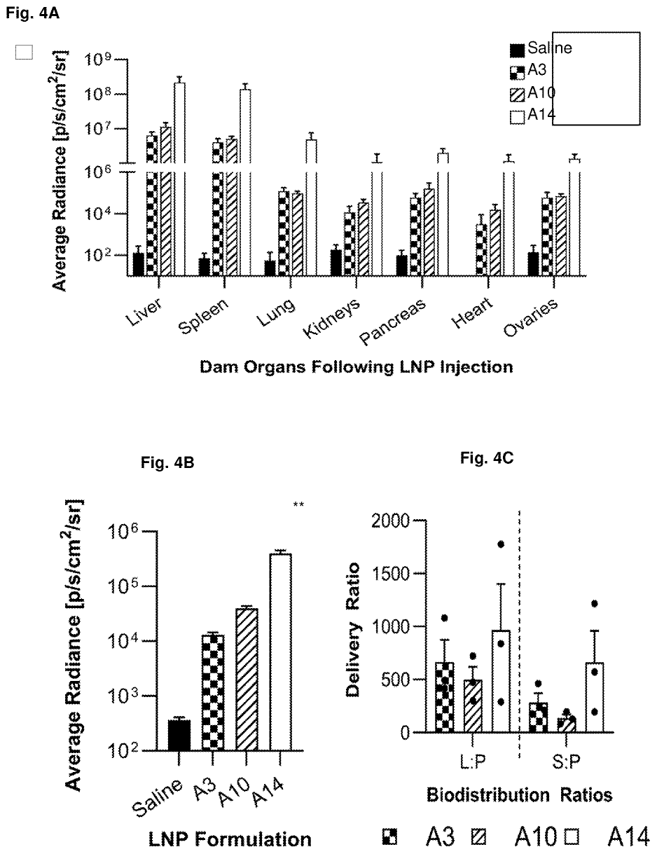

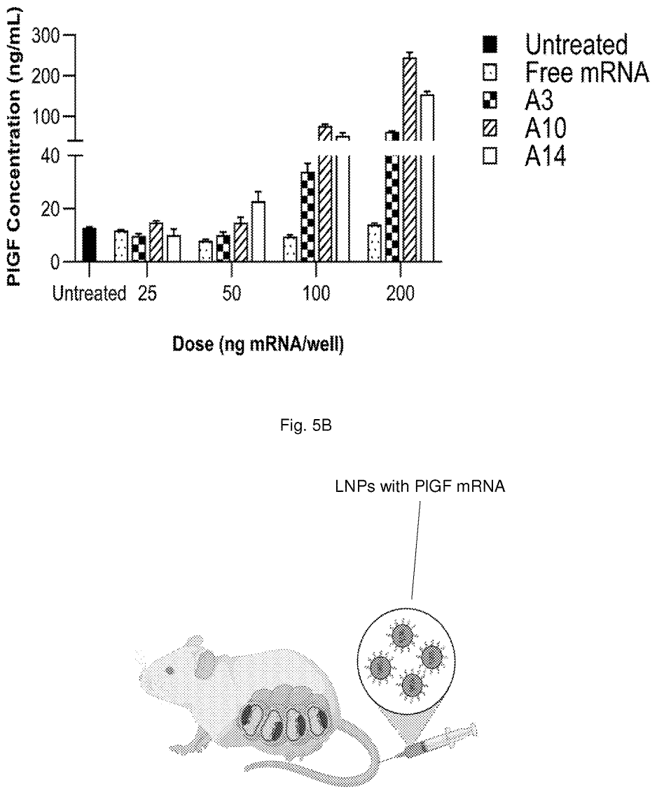

LNPs Deliver mRNA to Placentas Following IV Administration. Pregnant CD1 mice (dams) at embryonic day (E) 17.5 were injected with LNPs A3, A10, and A14 via the tail vein (0.5 mg mRNA/kg body weight). After 4 hours, the dam, placentas, fetuses, and maternal organs were imaged sequentially by IVIS. LNP A14 yielded the highest luminescence in the dam organs overall compared to dams treated with LNPs A3 and A10 (FIG. 4A). The liver and spleen had the highest and second highest luciferase expression, respectively, compared to all other dam organs, for all LNPs. This high level of liver and spleen delivery agrees with prior literature due to high blood flow to these tissues and apolipoprotein E-mediated uptake (Akinc, A. et al. Mol Ther 18, 1357-1364 (2010), Shi, B. et al. J Histochem Cytochem 59, 727-740 (2011)).

The placentas and fetuses collected from saline- or LNP-treated dams were imaged. Importantly, no LNPs resulted in detectable luciferase expression in the fetuses by IVIS. A nested mixed effects model revealed that LNP A14 had significantly higher luciferase expression in the placentas overall compared to LNPs A3 (p=0.031) and A10 (p=0.042) and the saline group (p=0.026, FIG. 4C). Of note, LNP A14 is the same formulation that was previously designed for high mRNA delivery to mouse livers (Kauffman, K. J. et al. Nano Lett 15, 7300-7306 (2015)). Thus, LNP A14 was expected to have the best delivery efficiency in the liver, which may be attributed to its less protonated surface (apparent pKa of 5.848) (Carrasco, M. J. et al. Commun Biol 4, 956 (2021)). However, the luciferase expression in the placenta following LNP A14 delivery contradicted our in vitro results that showed LNP A10 yielded significantly higher mRNA delivery in trophoblasts (FIG. 2F). As described above, the high luciferase expression from LNP A10 in vitro is due to a lower molar amount of PEG in the LNP formulation causing an increase in apparent pKa compared to LNP A14 (FIG. 3D). This same logic does not apply to an in vivo model, as studies have shown that PEG-lipids are desorbed from LNPs within two hours following systemic administration (Mui, B. L. et al. Mol Ther Nucleic Acids 2, e139 (2013)). This indicates that the amount of PEG may not influence delivery to the placenta in vivo. Thus, the findings correlating PEG molar ratio to apparent pKa appear to be relevant for in vitro delivery more so than in vivo systemic delivery. LNP delivery results to cells in vitro often do not correlate with delivery efficiency in vivo (Paunovska, K. et al. Nano Lett 18, 2148-2157 (2018), Whitehead, K. A. et al. ACS Nano 6, 6922-6929 (2012)). This limitation can be lessened by using primary human or mouse cells, rather than cell lines, and consistent LNP formulation techniques to improve in vitro and in vivo delivery correlation. Furthermore, the use of DOEs, such as the approach herein, and iterative library design allows for the collection of more in vitro data to predict which factors in the LNPs will have significant effects on in vivo efficacy.

To further elucidate the applicability of LNPs for mRNA delivery to the placenta, luciferase expression in the liver and spleen was directly compared to the placentas by calculating liver:placenta (L:P) and spleen:placenta (S:P) delivery ratios using the average radiance with background subtracted for each image. The L:P ratio was 1.9-fold lower, and the S:P ratio was 4.9-fold lower, in mice treated with LNP A10 compared to those treated with LNP A14 (FIG. 4D). While not statistically significant, this may indicate that LNP A10 is more efficient at delivering mRNA to the placenta relative to the liver and spleen compared to LNP A14. Thus, LNP A10 may be bias towards placenta delivery, which may limit off-target effects when treating placental dysfunction.

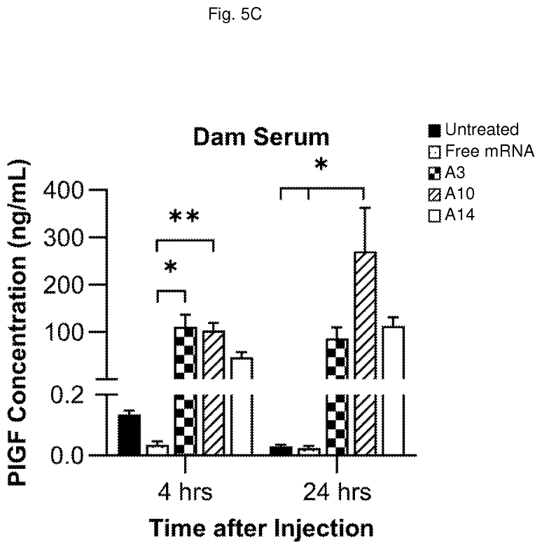

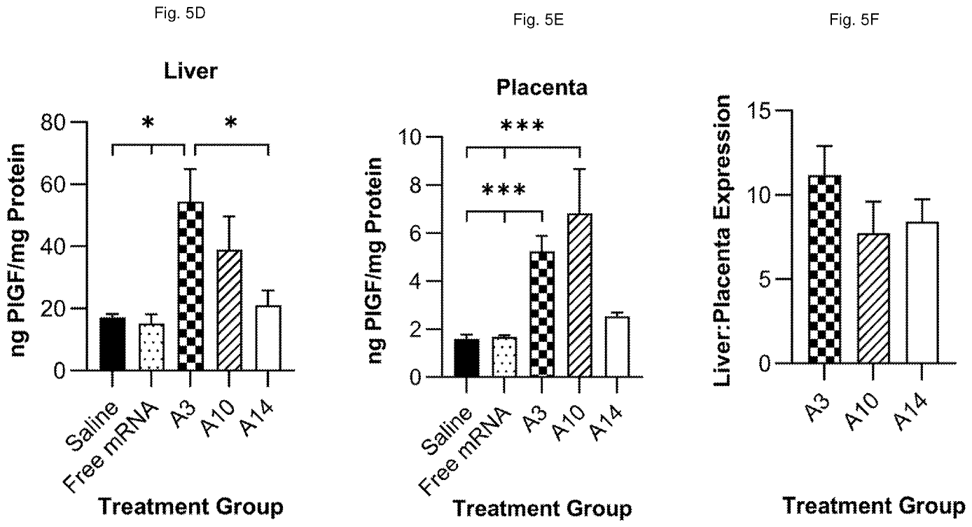

LNP-Mediated Delivery of PIGF mRNA. Next, placental delivery of these platforms using PIGF mRNA was evaluated as a more therapeutically-relevant mRNA to demonstrate induced protein synthesis and secretion from the placenta. As explained above, circulating PIGF levels are decreased in diseases of pregnancy, such as preeclampsia and fetal growth restriction, compared to a healthy pregnancy (Zeisler, H. et al N Engl J Med 374, 13-22 (2016), Agrawal, S., Shinar, S., Cerdeira, A. S., Redman, C. & Vatish, M. Hypertension 74, 1124-1135 (2019)). The reduced PIGF increases the sFlt1:PIGF ratio, contributing to decreased angiogenesis in the placenta that is found in preeclampsia (Verlohren, S. et al. Am J Obstet Gynecol 206, 58 e51-58 (2012), Zeisler, H. et al. N Engl J Med 374, 13-22 (2016), Levine, R. J. et al. N Engl J Med 350, 672-683 (2004)). Thus, LNPs were formulated with PIGF mRNA with the goal of increasing circulating PIGF and local PIGF expression in the placenta. The remainder of the data herein uses LNPs with encapsulated PIGF mRNA in place of the luciferase mRNA used in prior studies. We treated BeWos with LNPs A3, A10, or A14 and assessed secreted PIGF content after 24 hours. At most doses, BeWos treated with LNP A10 produced the highest PIGF levels compared to the other formulations and free mRNA (FIG. 5A). LNP A10 yielded 1.58-fold higher PIGF secretion compared to LNP A14 at a dose of 200 ng mRNA/well. This agrees with our in vitro results with luciferase mRNA (FIG. 2F-H), as it demonstrates that LNP A10 is the most efficient at delivering mRNA to BeWos in vitro.

PIGF mRNA delivery in vivo was also assessed. LNPs A3, A10, and A14 were injected via the tail vein (0.5 mg mRNA/kg body weight) (FIG. 5B). For these studies, we analyzed PIGF content in dam serum after 4 and 24 hours to track the secretion kinetics of PIGF over time. At 4 hours after injection, LNP A3 produced 111.4 ng/mL PIGF in the dam serum, compared to 103.6 and 46.6 ng/ml of PIGF produced following treatment with LNPs A10 and A14, respectively (FIG. 5C). Treatment with A3 (p=0.0219) and A10 (p=0.0069) had a statistically significant increase in PIGF in the serum compared to free mRNA at 4 hours (FIG. 5C). At 24 hours, LNP A10 produced 270.2 ng/ml of PIGF in the dam serum compared to 86.6 and 113.4 ng/ml of PIGF produced following treatment with LNPs A3 and A14, respectively (FIG. 5C). Treatment with LNP A10 yielded a statistically significant increase in PIGF in the serum compared to dams treated with saline (p=0.019) and free mRNA at 24 hours (p=0.013, FIG. 5C). The difference between serum concentration of PIGF at 4 and 24 hours was compared for each treatment group by a Wilcoxon test (Table S10.5). None of the treatment groups produced a statistically different amount of PIGF at 24 hours compared to 4 hours. However, it is noteworthy that LNP A10 yielded approximately 2.5× higher PIGF at 24 hours compared to 4 hours (p=0.125). Although not statistically significant, these data indicate that the LNP formulation may impact how quickly the mRNA is delivered and secreted from the placenta. Overall, our data does not demonstrate significant degradation or clearance of PIGF 24 hours after LNP delivery.

Although the data demonstrates that LNP A10 yields the highest level of PIGF secretion in dam serum, which tissues were generating the secreted PIGF was explored. Since the overall goal was to develop an LNP platform for placental delivery, the level of PIGF generated in the placenta and liver tissues were examined. The PIGF levels was compared in the liver because the prior data (FIG. 4A, B) demonstrated a high level of liver delivery. Dams treated with LNP A3 had the highest liver PIGF content with 54.4 ng of PIGF/mg of total protein (p<0.05, FIG. 5D). Alternatively, dams treated with LNP A10 had the highest PIGF content in the placenta with 6.81 ng PIGF per mg of total protein, which is 1.30-fold and 2.69-fold higher than LNPs A3 and A14, respectively (p<0.001, FIG. 5E). These results were consistent with in vitro results using PIGF mRNA, where LNP A10 had the highest PIGF secretion from BeWos (FIG. 5A). This data contradicts studies with luciferase mRNA at the time points studied, which found that LNP A14 yielded highest delivery overall in vivo compared to the other LNP formulations. This indicates that, in addition to LNP design, the mRNA sequence also plays a critical role for in vivo biodistribution.

A delivery ratio comparing PIGF levels in the liver to PIGF levels in the placenta demonstrated that LNP A10 exhibited the lowest liver:placenta (L:P) ratio that was 1.45-fold and 1.09-fold lower than LNPs A3 and A14, respectively (FIG. 5F). This result, combined with the higher serum PIGF content, indicates that LNP A10 is more efficient at delivering PIGF mRNA to the placenta compared to LNPs A3 and A14. Local placental delivery is important because PIGF levels in the placenta promote endothelial growth, vasculogenesis and overall placental development (Shah, D. A. & Khalil, R. A. Biochem Pharmacol 95, 211-226 (2015)). Evidence suggests the role of PIGF on endothelial-dependent relaxation mechanisms (Zhu, M., Ren, Z., Possomato-Vieira, J. S. & Khalil, R. A. Am J Physiol Regul Integr Comp Physiol 311, R505-521 (2016)) which may be advantageous locally in the placenta to improve uterine and placental vessel remodeling, while increasing blood flow to the fetus. Targeting ligands may be incorporated into this platform to further increase local placental delivery. Based on the data presented here, the LNP platform (A10) delivers multiple types of mRNA to the placenta.

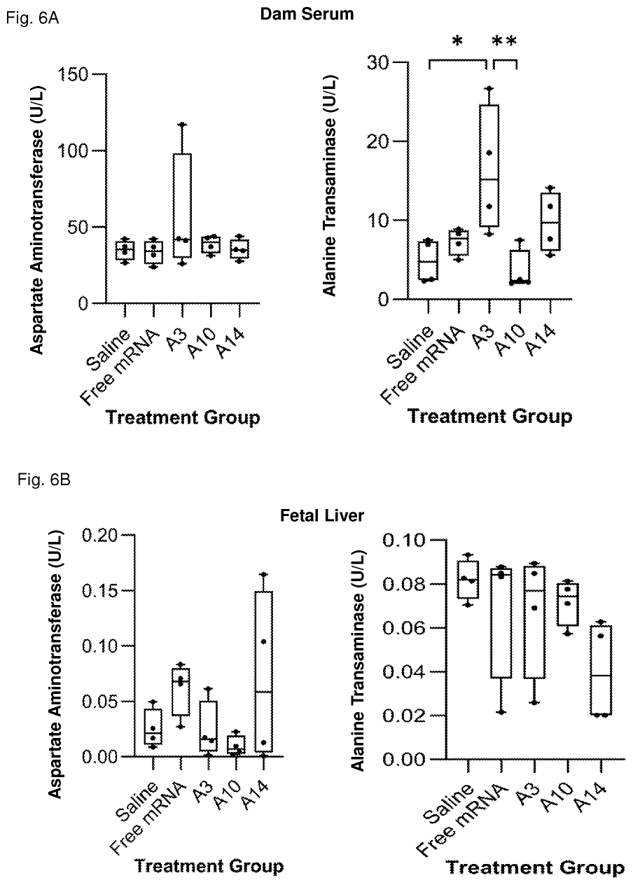

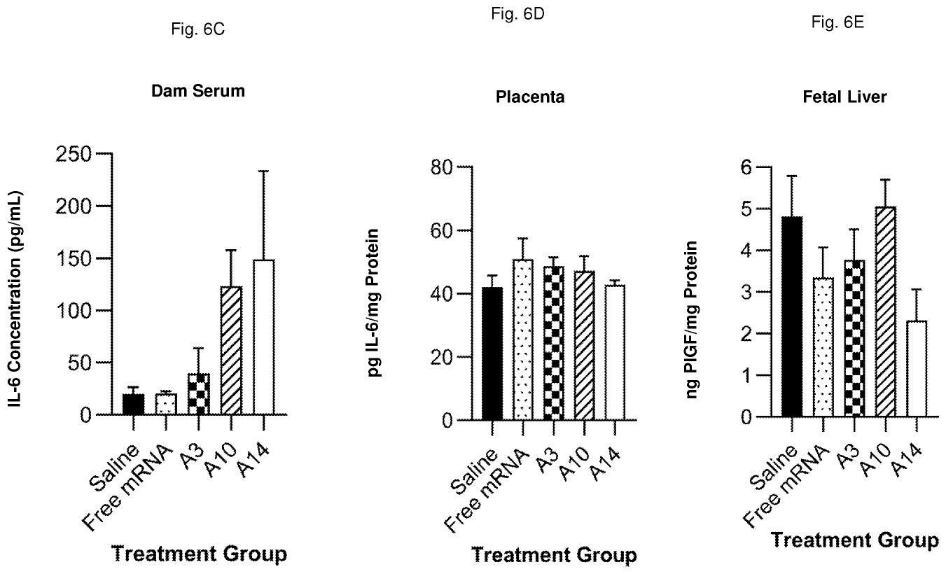

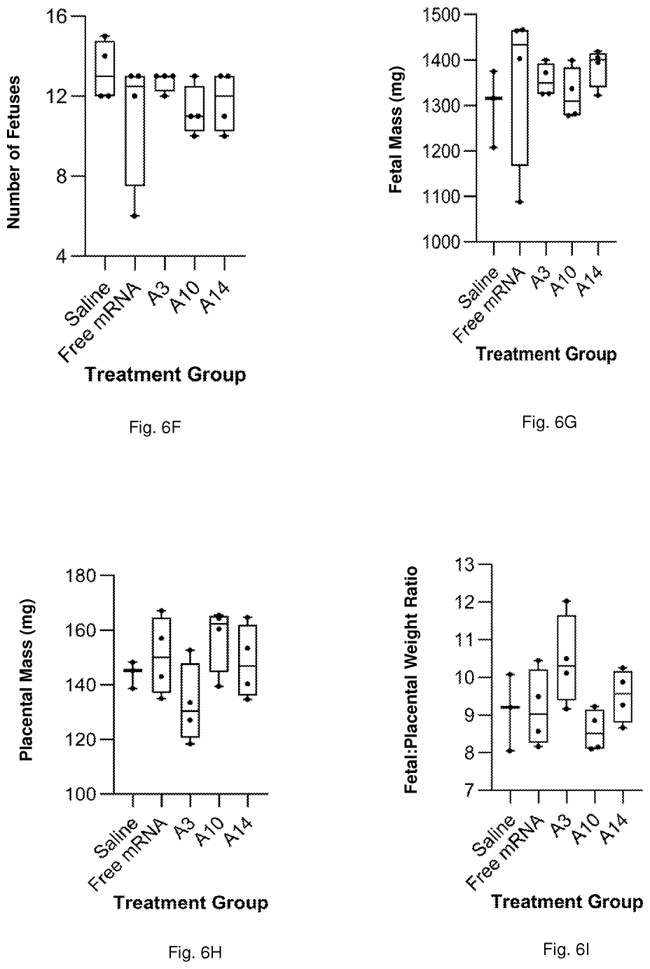

Toxicity Analysis. Lastly, toxicity of the LNPs was assessed with encapsulated PIGF mRNA to both the dams and fetuses. Serum from dams treated with LNPs A3, A10, and A14 was examined for aspartate aminotransferase (AST) and alanine transaminase (ALT) content to assess liver toxicity, which yielded no significant difference between AST levels in dam serum from all treatment groups (FIG. 6A). The only significant difference in serum ALT levels was from dams treated with LNP A3 compared to both saline (p=0.017) and LNP A10 (p=0.008, FIG. 6A). These results indicate that LNP A10, the top-performing LNP for PIGF delivery in vivo, does not yield liver damage as assessed by enzyme release in dams. AST and ALT content in fetal liver tissues following treatment was also assessed, which revealed no significant differences between fetuses from dams treated with each treatment group. Interestingly, two fetuses taken from dams treated with LNP A14 had slightly elevated AST (0.007 compared to 0.134 U/mg of total protein) that was not statistically significant (FIG. 6B).

The concentration of interleukin-6 (IL-6) in dam serum and placenta tissues 24 hours after treatment was measured to investigate the acute inflammatory response to LNPs (Parhiz, H. et al. J Control Release 344, 50-61 (2021). LNPs have been evaluated as adjuvants for vaccines because they induce IL-6 production (Alameh, M. G. et al. Immunity 54, 2877-2892 e2877 (2021)). IL-6 concentration in the dam serum was elevated following delivery of LNPs, compared to saline and free mRNA, but statistical analysis showed no significance between groups (FIG. 6C). Additionally, serum concentrations of IL-6 were below levels seen in previous studies of mice treated with LNPs containing mRNA (Parhiz, H. et al. J Control Release 344, 50-61 (2021), Alameh, M. G. et al. Immunity 54, 2877-2892 e2877 (2021), Parhiz, H. et al. J Control Release 291, 106-115 (2018)). The local inflammation in the placenta was also examined by measuring IL-6 concentration in digested placenta tissues, which yielded no differences between groups (FIG. 6D). This indicates that any changes in systemic IL-6 production do not originate from local immune activation in the placenta.

To evaluate potential fetal delivery of LNPs, PIGF content in the digested fetal liver tissues was measured (FIG. 6E). There was no significant difference between PIGF expression in the fetal livers from dams treated with LNPs compared to dams treated with saline, indicating no delivery to the fetus. As another measure for toxicity, the number of fetuses per dam at the time of dissection and tissue collection was averaged, which showed no significant difference between number of fetuses from all treatment groups (FIG. 6F). For this study, viability was determined based on fetal size as well as no visible tissue resorption. Any fetuses that were obviously resorbed were considered non-viable and were not included in any analysis. The fetuses and their respective placentas from each dam were weighed at the time of tissue collection, which revealed no difference between treatment groups (FIG. 6G-H). Using this data, a fetal to placental (F:P) weight ratio was calculated for each fetus and its placenta, which indicates the overall health of the fetus and placenta with no difference between treatment groups (FIG. 61) (Coan, P. M. et al. J Physiol 586, 4567-4576 (2008), Hayward, C. E. et al. Front Physiol 7, 28 (2016), Irvin-Choy, N. S., Nelson, K. M., Dang, M. N., Gleghorn, J. P. & Day, E. S. Nanomedicine 36, 102412 (2021)). Taken together, the toxicity analyses indicate that the top platform, LNP A10, is nontoxic to both the dams and the fetuses following treatment. These results, combined with the high luciferase and PIGF mRNA delivery, demonstrate that LNP A10 may serve as a potent and safe drug delivery platform for placenta-related diseases.

Research on placenta-related diseases has identified low PIGF as a clinical biomarker of preeclampsia and fetal growth restriction. However, limited studies have investigated PIGF as a protein replacement therapy to restore angiogenic factor balance for these diseases (Suzuki, H. et al. Hypertension 54, 1129-1135 (2009), Spradley, F. T. et al. Hypertension 67, 740-747 (2016), Makris, A. et al. Hypertension 67, 1263-1272 (2016). In mouse models of preeclampsia, intraperitoneal injection with recombinant mouse or human PIGF decreased arterial blood pressure and circulating sFlt-1 (Suzuki, H. et al. Hypertension 54, 1129-1135 (2009), Spradley, F. T. et al. Hypertension 67, 740-747 (2016)). Subcutaneous injection with recombinant human PIGF into nonhuman primates with surgically induced uteroplacental ischemia decreased blood pressure, proteinuria, and sFlt-1 mRNA expression in the placenta Makris, A. et al. Hypertension 67, 1263-1272 (2016)). These studies showed that increased circulating PIGF improved clinical outcomes in animal models of preeclampsia, validating its potential use as a therapeutic.

Normal serum levels of PIGF in humans varies based on gestational age, peaking around 30 weeks in the third trimester. Below a serum PIGF level cutoff between 80 and 120 μg/mL is considered predictive of adverse pregnancy outcomes (Agrawal, S., Shinar, S., Cerdeira, A. S., Redman, C. & Vatish, M. Hypertension 74, 1124-1135 (2019)). Patients with low serum PIGF levels (<100 μg/mL) at the time of testing (20 to 35 weeks of gestation) were 58.2% more likely to develop early-onset preeclampsia (<34 weeks of gestation) (McLaughlin, K. et al. Hypertension 77, 2057-2065 (2021), Kluivers, A. C. M. et al. Ultrasound Obstet Gynecol (2022)). The results shown here demonstrated that LNPs have the potential to produce PIGF secretion in vivo at levels much greater than what is seen in human pregnancy. Our top formulation, LNP A10, yielded approximately two orders of magnitude higher PIGF levels in the dam serum compared to what is typically seen during pregnancy (~160-1800 μg/ml) (Saffer, C. et al. Pregnancy Hypertens 3, 124-132 (2013)). This indicates that further studies regarding the dosing would be warranted to potentially lower the administered dose to achieve normal levels. However, it is important to note that the physiological differences between mouse and human pregnancy would likely contribute to the level of PIGF secretion observed. For example, mice in these studies carried up to 15 fetuses in a pregnancy, whereas the majority of human pregnancies have one fetus and placenta. Thus, the high level of PIGF secretion may be a result of multiple placentas secreting PIGF. Directly corresponding these results to human pregnancy will require further studies in larger animal models, such as sheep or non-human primates. In the future, the top LNP formulations will be used in preeclamptic animal models to assess the impact of PIGF mRNA delivery in placenta pathology and function.

There are a few off-target effects of administering PIGF to consider. For example, constitutively expressed PIGF in a transgenic mouse model yielded enhanced vessel permeability (Odorisio, T. et al. Journal of Cell Science 115, 2559-2567 (2002)) and inhibition of apoptosis (Adini, A., Kornaga, T., Firoozbakht, F. & Benjamin, L. E. Cancer Research 62, 2749-2752 (2002)). The LNP platform described herein overcomes these off-target effects because protein expression following mRNA delivery is transient. The short half-life of mRNA is a major benefit of this platform during pregnancy. The goal of disease management during pregnancy, as described here, is to extend pregnancy several weeks to reduce the risks of preterm birth. Since the goal is not permanent gene therapy, many of the long-term risks associated with PIGF administration are alleviated. Although the LNPs described here, similar to other nanoparticle delivery systems, yield liver delivery, our top LNP A10 resulted in the lowest liver:placenta ratio compared to the other formulations tested. In addition, targeting ligands may be incorporated into LNPs to improve placental targeting and minimize off-target effects to maternal tissues. Finally, the biodistribution results using luciferase mRNA encapsulated in LNPs demonstrated no delivery of LNPs to the fetus. This, combined with the toxicity analysis, suggests no adverse effects of LNPs to fetuses. These results support the use of LNPs, and in particular, LNP A10, for mRNA delivery to the placenta to treat diseases that originate from placental dysfunction.

### Example 2

Preeclampsia is the most common disease of pregnancy, affecting 5-8% of pregnancies worldwide. Preeclampsia is diagnosed in the second trimester of pregnancy and is characterized by new onset, persistent hypertension with evidence of end organ damage, most commonly proteinuria. While the pathophysiology remains to be fully elucidated, evidence suggests that there is an immune mismatch at the maternal-fetal border that propagates poor spiral artery invasion into the myometrium; this leads to systemic hypertension and other symptoms of preeclampsia. A driving mechanism of this immune dysfunction is dysregulation of macrophage phenotypes within the placenta. Specifically, there is an aberrant increase in proinflammatory (M1) macrophages and a decrease in tolerogenic (M2) macrophages compared to healthy pregnancies. Decreasing the M1:M2 ratio in preeclamptic placentae may yield a tolerogenic environment at the maternal-fetal border to promote spiral artery invasion and remodeling, thus preventing or reversing the hypertensive effects. Mechanistically, cytokines present in the local environment, including interleukin-4 (IL4) and interleukin-13 (IL13) polarize macrophages towards an M2 phenotype, characterized by increased surface expression of CD206 and CD209. While IL4 and IL13 delivery provides a means to promote tolerogenic M2 macrophages, systemic cytokine delivery leads to widespread toxicity due to excess immune activation. Here, this challenge was overcome and the therapeutic potential of these cytokines using ionizable lipid nanoparticles (LNPs) demonstrated.

LNPs were developed encapsulating IL4 or IL13 mRNA that deliver to the placenta and transfect placental cells, called trophoblasts, leading to cytokine secretion within the placenta. The data indicated that the LNPs successfully transfected trophoblasts in vitro and in vivo. Further, sufficient secretion of IL4 and IL13 was achieved from trophoblasts to polarize macrophages to an M2 phenotype in vitro, as indicated by CD209/CD206 expression and IL10 secretion. Taken together, these results demonstrated the potential for LNPs to be used as tools for (1) local immune modulation in the placenta to treat preeclampsia, and (2) to study the role of the local immune system in preeclampsia onset and progression.

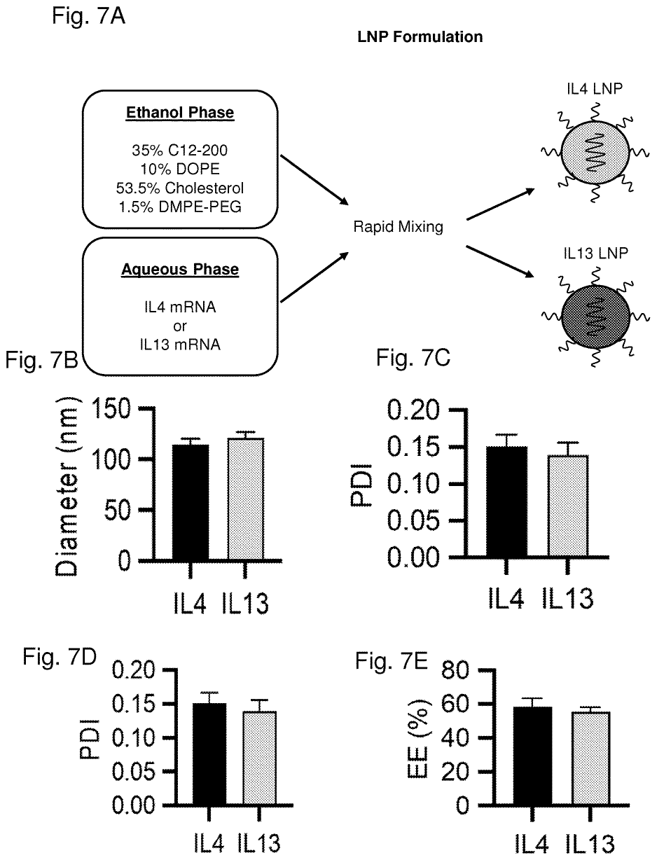

LNP Fabrication and Characterization: The lipid nanoparticle (LNP) formulation was previously determined using a Design of Experiments approach.¹ (Example 1; FIG. 7A) Briefly, a library of LNPs were screened to determine the highest performing formulation for the delivery of mRNA to placental cells as previously described.¹ LNPs were formulated using a four-component system consisting of an aqueous phase and an organic phase. The aqueous phase consisted of pH 3.0 citrate buffer and 1-methylpseudouridine modified mRNA. The organic phase contained C12-200 (Ionizable lipid), 1,2-dioleoyl-sn-glycero-3-phosphoethanolamine (DOPE) (Phospholipid), Cholesterol, and DMPE-PEG2000 (PEG lipid). (FIG. 1B) The mRNA was modified to reduce toll-like receptor (TLR) activation after LNP transfection.² The ionizable lipid was the functional component of the LNP formulation, taking on a positive charge when mixed with the acidic citrate buffer. This allowed for the complexation of the negatively charged mRNA molecules with the protonated ionizable lipids.³ The other lipid components are necessary for the stability and solubility of the LNPs allowing for effective delivery of mRNA in aqueous mediums.³ A lipid nanoparticle formulation was designed for preferential mRNA delivery to mouse placentas. Using this LNP design mRNA sequences coding for human IL4 (hIL4), human IL13 (hIL13), murine IL4 (mIL4), murine IL13 (mIL13), or firefly luciferase (Luc) were encapsulated. LNPs for size by dynamic light scattering, which resulted in sizes ranging from 103.5-136.9 nm and low polydispersity indices <0.21 (FIG. 7B, C). Average mRNA encapsulation ranged from 44.19-72.03% (FIG. 7D), as assessed by Ribogreen assays.

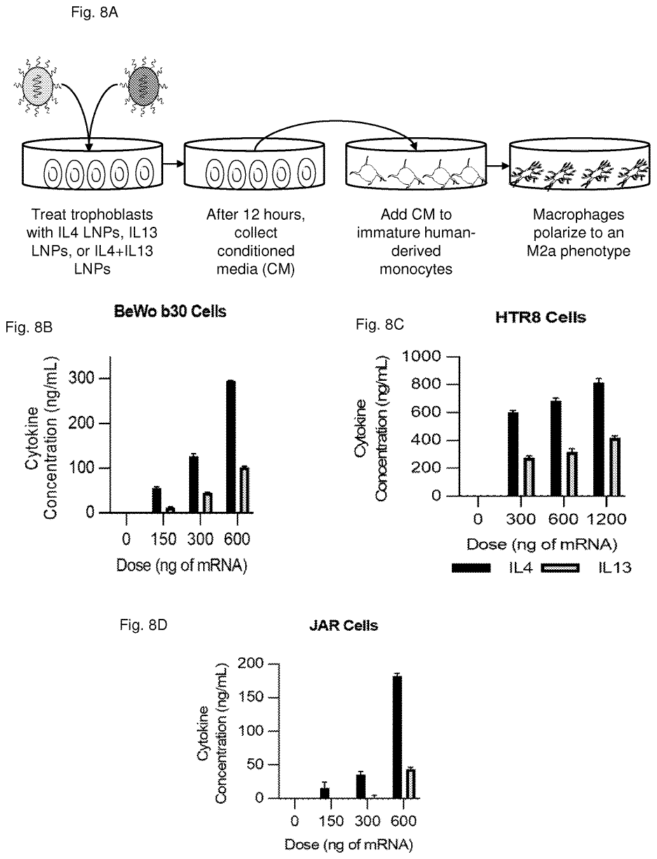

In vitro LNP Delivery to Placental Cell Lines: After successful LNP formation and mRNA encapsulation was confirmed, in vitro LNP delivery was assessed across 3 separate placental cell lines. BeWo b30 (BeWo) cells are a choriocarcinoma cell line that has been extensively used to study placental pathologies, and are the most commonly studied cell line for diseases of the placenta.^{4,5} HTR8/svNEO (HTR8) cells are immortalized first trimester trophoblast cells and JAR cells are another choriocarcinoma cell line.⁵ Both HTR8 and JAR cells were chosen to demonstrate the ability of our LNP formulations to transfect and stimulate the ectopic secretion of cytokine across multiple cell lines, modeling different stages of placental development. All three cell lines demonstrated a dose response when the dose of encapsulated mRNA was modulated. When treated with 0, 150, 300, or 600 ng of total mRNA, BeWos secreted 0, 55.75, 126.82, and 295.28 ng of IL4 and 0, 12.17, 45.44, and 102.37 ng of IL13 respectively. When treated with 0, 300, 600, or 1200 ng of total mRNA, HTR8s secreted 0, 601.22, 680.44, and 813.28 ng of IL4 and 0, 274.95, 318.98, and 418.37 ng of IL13 respectively. When treated with 0, 150, 300, or 600 ng of total mRNA, JARs secreted 0, 15.68, 35.53, and 181.67 ng of IL4 respectively. Only in the 600 ng dose of mRNA was there any IL13 secretion (43.46 ng). From this data, it was determined that BeWos were transfected the most efficiently by LNPs as demonstrated by the enhanced cytokine secretion at lower doses than the HTR8 cells. This data also suggests that HTR8s may be the most resistant to LNPs mediated cytotoxicity as lower doses needed to be used to treat both BeWo and JAR cells due to observed toxicity. (FIG. 8)

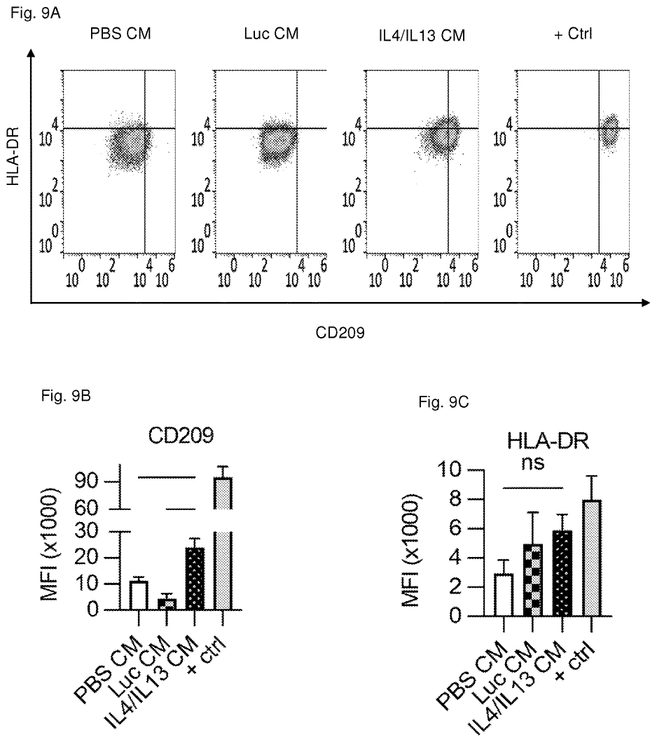

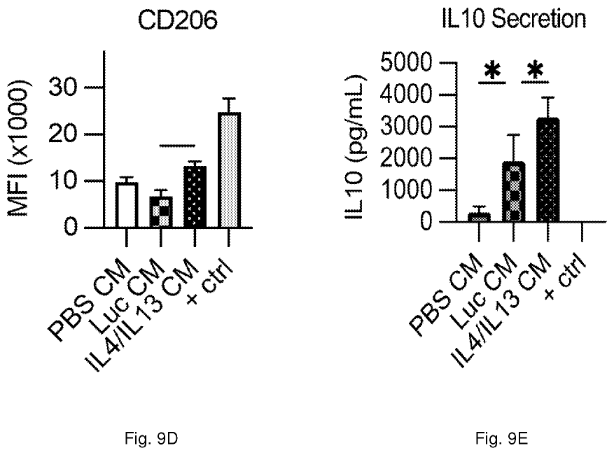

In vitro macrophage polarization from LNP conditioned media: The remainder of in vitro experiments were performed in BeWos. BeWos were treated with 5000 ng/flask of mRNA in T25 flasks. This dose was chosen as it is analogous to a 100 ng/ml dose from the experiments performed in FIG. 8. Doses of 40 ng/mL of IL4 and 20 ng/ml of IL13 have been established in the literature to successfully polarize macrophages to an alternatively activated M2 fate.⁵ Extrapolating results from the dose response experiments shown in FIG. 8, a dose of 5000 ng/flask was expected to produce conditioned media (CM) cytokine concentrations of approximately 40 ng/ml IL4 and 20 ng/ml of IL13 allowing for the direct comparison of cytokine treated groups to recombinant peptide treated positive control groups. CM treated CD14⁺CD11b⁻ macrophages exhibited a marked upregulation of the M2 marker, CD209 (DC-SIGN) expression. (FIG. 9A,B) There was a significant increase in CD209 expression in the IL4/IL13 CM treated groups when compared to the PBS CM group which received an equal volume of PBS in place of LNPs to act as a negative control. (FIG. 9A,B) Additionally, there was a significant increase in CD209 expression in the IL4/IL13 CM group when compared to an equivalent mRNA dose of Luc-LNPs. (FIG. 9A,B) There was no significant change in HLA-DR expression across any of the groups tested. (FIG. 9A,C) Further analysis of the CD14⁺CD11b⁻HLA-DR⁻ population elucidates the changes in the expression of another marker of alternatively activated M2 macrophages, CD206. It was determined that there is no significant difference in CD206 expression of CD14⁺CD11b⁻HLA-DR⁻ cells between the PBS CM and Luciferase CM groups. While it was demonstrated that there is a significant increase in CD206 expression when compared to both PBS CM and Luc CM negative controls as assessed by increased mean fluorescence intensity (MFI). (FIG. 9D) The CD206 expression was significantly higher in the positive control group which was treated with recombinant peptides at the concentrations mentioned previously. (FIG. 9D)

In addition to determining the changes in the expression of surface markers, functional assessment of the macrophages was determined after CM treatment. IL10 secretion is a hallmark of the alternatively activated macrophage phenotype.^{6,7} PBS CM elicited a relatively minor increase in IL10 secretion from macrophages (303.06±192.92 μg/mL). Whereas exposure to Luciferase CM elicited a significantly larger IL10 response from macrophages (1513.32±340.75 μg/mL). As expected, exposure to IL4/IL13 CM elicited the largest IL10 response (3452.95±808.64 μg/mL) significantly larger than all other groups. Interestingly, the macrophages exposed to the positive control peptide media did not secrete any detectable IL10 when assessed by ELISA. (FIG. 9E)

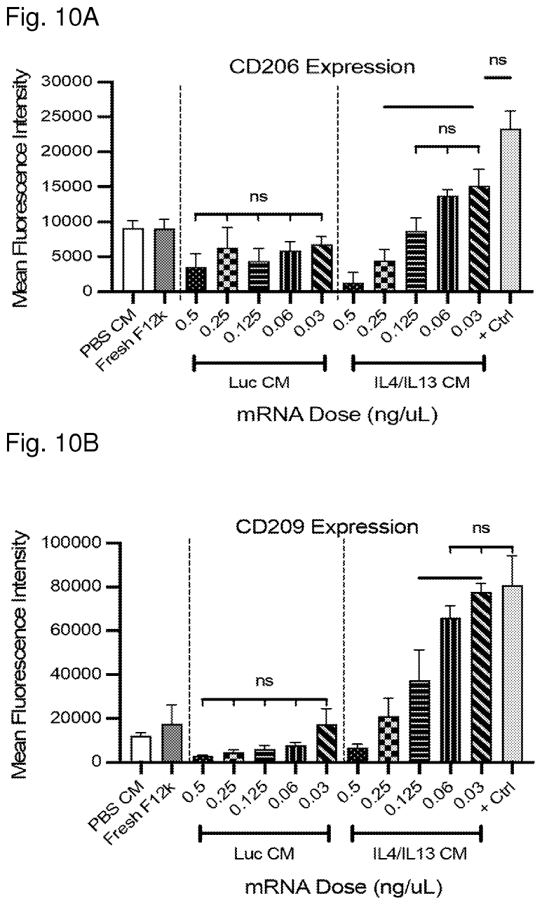

It has been suggested that post-translational modifications have a significant impact on the potency and efficacy of protein-based drugs.^{8,9} After completing the previous in vitro experiments at a dose of 1.0 ng/μL of total mRNA which corresponded to approximately the same peptide concentration as the positive control groups (40 ng/ml IL4 and 20 ng/mL IL13) it was observed that the macrophage polarization was much lower than expected. The dose of mRNA delivered was modulated to elucidate if that would play a role in increasing the expression of surface markers for M2 polarization. Since the chosen dose was at the bottom of the range of tested doses in our dose response experiments (FIG. 8), it was known that BeWo cells could produce significantly more cytokine if given a higher dose of LNPs. The LNP dose was increased to increase the concentration of cytokine in the resultant CM. When macrophages were cultured in CM with higher cytokine concentrations, surface marker expression decreased or remained the same. A broader dose response curve was performed with 0.5 ng/μL as the highest dose delivered to macrophages and the doses were lowered by half ranging from 0.5-0.03 ng/μL. (FIG. 10). Interestingly, as the dose of LNPs was decreased the surface expression of CD209 and CD206 increased significantly in CD14⁺CD11b⁻ macrophages. (FIG. 10). As the dose of delivered mRNA decreased to 0.06 ng/μL the difference in expression of CD209 became statistically non-significant from the peptide controls cited previously. (FIG. 10A) When the dose was lowered to 0.03 ng/μL the difference in surface expression of both CD209 in CD14⁺CD11b⁻ macrophages (FIG. 10A) and CD206 in CD14⁺CD11b⁻HLA-DR⁻ macrophages (FIG. 10B) became statistically similar to that of peptide controls. This suggested that the cytokines produced in mammalian cells after LNP transfection are far more potent than recombinant peptides made in E. Coli bacterial systems.

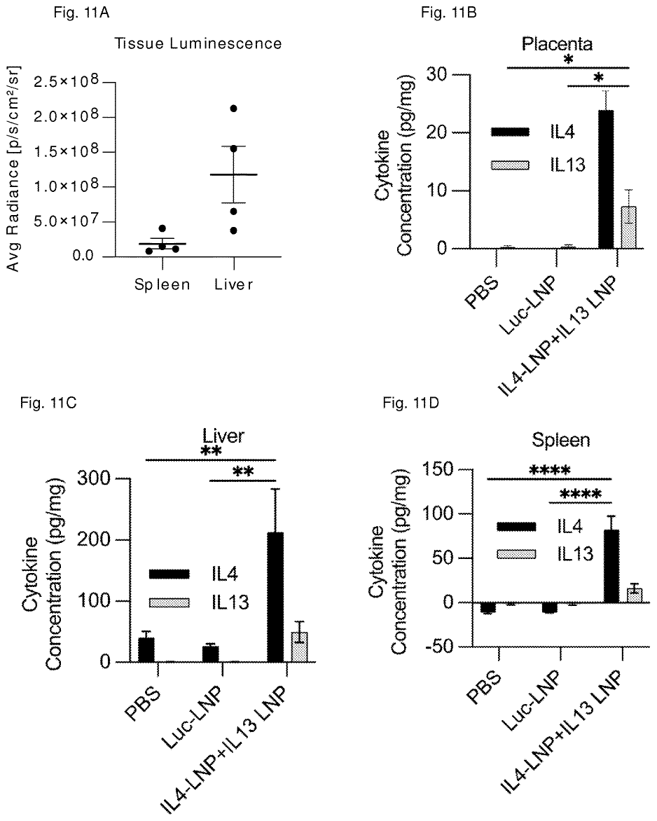

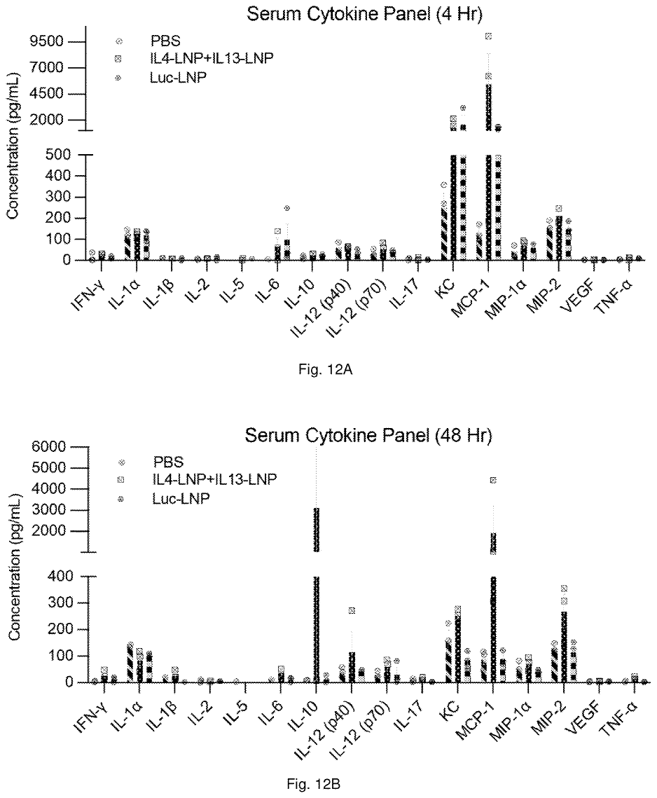

In vivo LNP biodistribution and macrophage polarization: For in vivo LNP assessment pregnant female CD-1 mice were injected via tail vein injection with 0.5 mg/kg of LNP encapsulated mRNA. Biodistribution studies were performed by injecting Luc-LNPs into the tail veins of healthy pregnant mice at gestational day 12.5 (E12.5). After 4 hours, D-luciferin was injected intraperitoneally into mice before being imaged using IVIS. (FIG. 11) Whole mouse images of live mice showed all luciferase activity in the abdomen of the mouse, primarily in the organs in and around the liver. After euthanizing the mice, they were dissected, and individual organs were isolated and repeat IVIS imaging was performed. After dissection, it was discovered that the majority of the luciferase expression was found in the liver and spleen of treated mice. (FIG. 11) Due to the high signal in the liver and spleen, luminescence was not observed in any other organs. (FIG. 11) To overcome the comparatively high signal in the liver and spleen, the uterine horn was isolated from the other organs. Placentae and fetuses were removed and separated from the uterine horn and then imaged separately. Placentas from dams treated with PBS and Luc-LNPs had no detectable IL4 or IL13 expression, indicating that any amount of these cytokines is a result of LNP-mediated delivery. Comparatively, dams treated with IL4-LNPs and IL13-LNPs exhibited significantly increased IL4 (~24 μg/mg) and IL13 (~7 μg/mg) in the placentae compared to controls. This data demonstrates that LNPs reach the placenta in sufficient levels at E12.5 to induce cytokine secretion. (FIG. 11) No luciferase expression was present in the fetuses of treated mice indicating that Luc-LNPs did not cross the placental border into the fetal space after systemic tail vein injection.

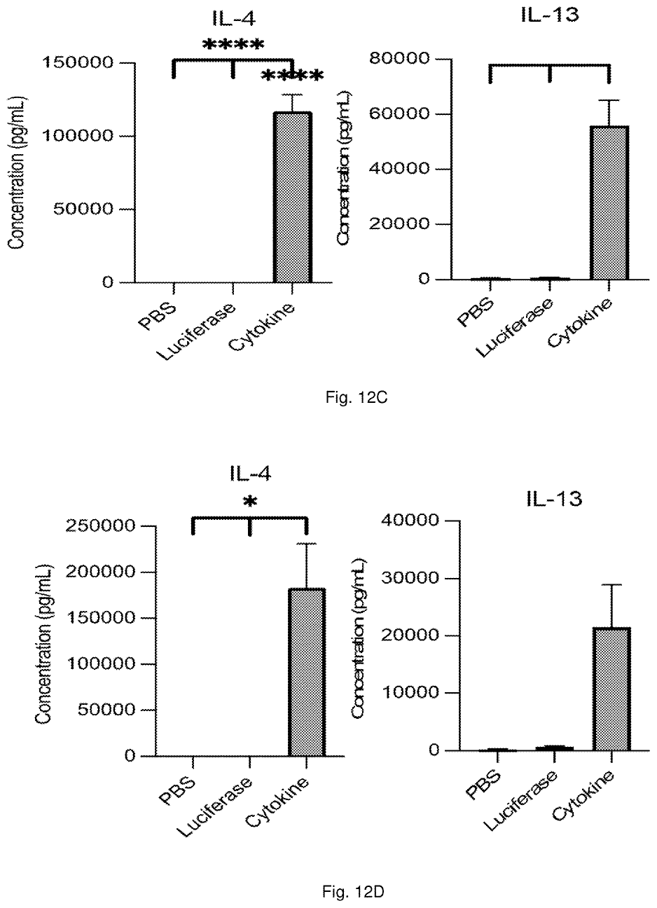

After imaging, samples were frozen at −80 °C until ELISAs were performed to determine cytokine levels in Luc-LNP treated mice. Concurrently, healthy pregnant CD-1 mice were injected with LNPs encapsulating mIL4 and mIL13 mRNA, in addition to an equal volume of PBS as a negative control. Tissue samples were collected and frozen at −80 °C until ELISAs were performed. After tissue digestion and protein extraction, the cytokine concentrations in the liver, placenta, and spleen were assessed. These organs were chosen as they had the highest delivery of Luc-LNPs as demonstrated in FIG. 11. For all tissues assessed, the IL13 levels were significantly lower than the IL4 levels which is consistent with the results from our in vitro experiments performed. (FIG. 8) As expected based on the results of the luciferase biodistribution study, the liver showed the highest IL4 and IL13 levels 4 hours after tail vein injection. (FIG. 10A) Consistent with the results of the biodistribution study (FIG. 11), the spleen demonstrated the second highest IL4 and IL13 levels after LNP injection. (FIG. 11A) While the placental levels of IL4 and IL13 were significantly lower than those found in the livers and spleens of treated mice, mice treated with IL4-LNPs and IL13-LNPs exhibited significantly higher cytokine levels than both PBS and Luc-LNP treated mice (FIG. 12B). Taken together, though delivery to the liver and spleen were significantly higher than delivery to the placentae, our data indicated there is preferential uptake to the placenta after IV injection compared to other tissues.

To evaluate the immunomodulatory and immunotoxicity effects of the LNPs, expression levels of various cytokines and chemokines in the serum 4- and 48-hr post-injection were measured by Luminex. Notably, IL-6, IL-1a, and TNF-α did not change between treatment groups at either timepoint, suggesting that the LNPs did not elicit significant systemic pro-inflammatory responses (FIG. 12). In contrast, the data revealed increased MCP-1 4-hours post-injection, and increased IL10 and MCP-1 48-hours post-injection from dams treated with IL4-LNPs and IL13-LNPs compared to controls (FIG. 12). MCP-1 is a potent chemoattractant responsible for recruiting monocytes to sites of tissue remodeling or inflammation (Yoshimura, T. Cell Mol Immunol 15, 335-345, (2018)). Alternatively, IL10 is an anti-inflammatory cytokine crucial for resolving inflammation and promoting tissue repair (Shapouri-Moghaddam, A. et al. J Cell Physiol 233, 6425-6440, (2018). The elevation of IL10 at 48-hr post-injection aligns with our in vitro data showing that polarized macrophages secrete IL10 (FIG. 9E), and is consistent with the expected effects of the delivering IL4 and IL13 mRNA. This suggests that LNP-mediated IL4 and IL13 delivery initiated anti-inflammatory macrophage polarization systemically in vivo. Together, these results indicate that while the LNPs did not provoke a classical pro-inflammatory cytokine response, they effectively promoted expression of chemokines important for immune cell recruitment, as well as the induction of IL10, supporting the anti-inflammatory and tissue-repairing functions of the delivered IL4 and IL13 mRNA.

Using LNPs to deliver cytokine mRNA, rather than recombinant peptides, offers an alternative approach for local cytokine therapy both during pregnancy and in other diseases. The importance of enabling local immunomodulation in the placenta is two-fold. First, controlling the immune state at the maternal-fetal border early in pregnancy could serve as a vaccine to support healthy pregnancy establishment and placenta development. Further, enabling immunomodulation in the placenta provides opportunity to study the role of the individual immune pathways in healthy and pathologic pregnancy establishment. More broadly, this approach to local immunomodulation can be exploited for other tissues as well to study underlying immunological pathways across a myriad of diseases.

LNP Formulation: The C12-200 ionizable lipid was fabricated in house. Other LNP components including cholesterol, DOPE, and DMPE-PEG2000 (ammonium salt)) were purchased from Avanti Polar Lipids Inc. (Birmingham, AL). Codon optimized mRNA was prepared by in vitro transcription through a collaboration with the Engineered mRNA and Targeted Nanomedicine core facility at the University of Pennsylvania (Philadelphia, PA). Human IL4, human IL13, mouse IL4, and mouse IL13 were synthesized with 1-methylpseudouridine modifications, co-transcriptionally capped using the CleanCap system (TriLink), and purified using cellulose based chromatography.¹⁰ LNPs were formulated by combining one volume of an organic phase containing the lipid components in ethanol and three volumes of the aqueous phase containing mRNA by rapid mixing with micropipettes. mRNA was diluted in citrate buffer (pH 3) to an mRNA:ionizable lipid weight ratio of 1:10 for all LNP formulations. The LNPs consisted of 53.5% cholesterol, 35% C12-200, 10% DOPE, and 1.5% DSPE-PEG-2000 by molar weight ratio. After mixing, the LNPs were dialyzed against PBS (pH 7.2) for 1 hour, sterile filtered using 0.2 μm filters, and stored at 4 °C.

LNP Characterization: Dynamic light scattering (DLS) measurements and mRNA encapsulation efficiency were assessed as previously described.¹ The average and standard deviation of the hydrodynamic diameter, polydispersity index, and encapsulation efficiency of each LNP is reported. For DLS, each LNP formulation was diluted 1:100 in deionized water in cuvettes and measurements were run on a Malvern Zetasizer Nano ZS (Malvern Panalytical). Encapsulation efficiency was calculated using the QuantiFluor® RNA System (Promega). Briefly, LNPs were diluted 1:100 in 1×Tris-EDTA (TE) buffer in two microcentrifuge tubes for each LNP formulation. 1% v/v Triton X-100 (Thermo Scientific) was added to one tube and both were heated to 37 °C and shaken at 600 RPM for 1 min, followed by cooling to room temperature for 10 mins. LNP samples and RNA standards were plated in triplicate in black 96-well plates, the fluorescent reagent was added, and intensity was measured using a plate reader (Molecular Devices) per the manufacturer instructions (excitation, 492 nm; emission, 540 nm). Background signal was subtracted from each well and triplicate wells for each LNP were averaged. RNA content was quantified by comparing samples to the standard curve, and encapsulation efficiency (%) was calculated according to the equation below, where A is the RNA content in samples without Triton X-100 treatment (intact LNPs) and B is the RNA content in samples with Triton X-100 (lysed LNPs).

$$E E = \frac{B - A}{B} \times 100$$

Cell Culture: BeWo b30 subclone (BeWo) cells were cultured in Kaighn's F-12k with L-glutamine (F12k) media (Corning) supplemented with 10% fetal bovine serum (Avantor) and 1% penicillin/streptomycin (pen/strep, VWR). HTR8/svNEO and BeWos were cultured in either 24-well plates or 25 cm² (T25) flasks at plating densities of 1.2×10⁵ cells/well or 1.0×10⁶ cells/flask, respectively. Peripheral human monocytes collected from healthy donors were purchased from The Human Immunology Core at The University of Pennsylvania and cultured in RPMI-1640 media supplemented with 10% fetal bovine serum and 1% penicillin/streptomycin in 24-well plates. Cultures were grown in an incubator set at 37 °C with 5% CO₂.

LNP Delivery to Trophoblasts in vitro: BeWo, HTR8/svNEO, and JAR cells were plated at 1.2e5 cells/well in 24-well plates and incubated for 6 hours to adhere. After 6 hours, IL-4-LNPs or IL-13-LNPs were added into each well using a micropipette and gently mixed by lateral translation of the plate. Treated cells were incubated for 24 hours, after which media containing secreted cytokines was collected and stored in microcentrifuge tubes (Thomas Scientific) at −80 °C for quantification by ELISA. Cytokine content in samples was assessed using IL-4, IL-13, and IL-10 ELISA assays (ThermoFisher). Samples were thawed on ice, mixed, and diluted in ELISA assay buffer. Appropriate dilutions were determined by serially diluting samples 0-1000-fold in assay buffer. Samples of cytokine-containing media were prepared following manufacturer instructions for each ELISA kit and absorbance was measured using a plate reader at 450 nm. Absorbance was compared to a standard curve to quantify the amount of cytokine in each sample. Statistical analysis was performed using GraphPad. One-way ANOVA with posthoc Tukey *p<0.05, **p<0.01, ***p<0.001. ****p<0.0001.

Macrophage Polarization: Human monocytes were cultured at 2.0×10⁶ cells/well in a 24-well plate in RPMI-1640 supplemented with macrophage colony stimulating factor (M-CSF), FBS, and pen/strep. Media was exchanged with fresh media after 72 hours, and the cells were cultured for a total of 5 days prior to experiments described below. Concurrently, BeWos were seeded at 1.0×10⁶ cells/flask in 25 cm² (T25) flasks in F12k media and allowed to adhere for 8 hours. After 8 hours, BeWos were treated with PBS, luc-LNPs, IL-4-LNPs, IL-13-LNPs, or equal doses of IL-4-LNPs and IL-13-LNPs at a dose of 0.5 ng/μL total mRNA for 12 hours (FIG. 7). After 12 hours, 1.0 mL of conditioned media from LNP-treated BeWos, or media containing IL-4 (40 ng/ml) or IL-13 (20 ng/ml) recombinant peptides as controls, was transferred onto the monocytes. Unless otherwise noted, all treatment groups included M-CSF (40 ng/ml) (PeproTech) throughout the experiments. Lastly, macrophage polarization was assessed by flow cytometry, as explained below. Statistical analysis was performed using GraphPad. One-way ANOVA with posthoc Tukey *p<0.05, **p<0.01, ***p<0.001, ****p<0.0001.

Biodistribution Studies in Pregnant Mice: Female CD-1 mice between 8-39 weeks of age were maintained, bred, and used in accordance with Animal Use Protocols approved by the Institutional Animal Care and Use Committee at The University of Delaware (AUP #1320 and #1341). Mice at gestational day (E) 12.5 were intravenously injected via the tail vein with luc-LNPs at a dose of 0.5 mg mRNA/kg mouse weight. After 4 hours, dams were anesthetized and injected intraperitoneally with d-luciferin potassium salt (150 mg/kg) (Biotium). Anesthetized dams were placed supine into the in vivo imaging system (IVIS) (Lumina III, PerkinElmer). After whole body imaging, dams were euthanized and the placentae, fetus, liver, lung, endometrium, heart, brain, and spleen were collected and imaged as described above. Raw luminescence (radiance) is reported which is a measure of the total photons released in each sample with the units (photons/sec/cm²/sr). Due to the nature of the measurements, normalization of data was not necessary.

LNP Delivery in vivo: Female mice between 8-39 weeks of age were maintained, bred, and used in accordance with Animal Use Protocols approved by the Institutional Animal Care and Use Committee at The University of Delaware (AUP #1320 and #1341). Mice at E12.5 were intravenously injected via the tail vein with 0.5 mg mRNA/kg mouse of IL-4-LNPs and IL-13-LNPs, luc-LNPs. Control mice were treated with sterile PBS. After 4 hours, mice were euthanized and the placentae, fetus, liver, lung, endometrium, heart, brain, and spleen were collected. Whole blood was obtained by ventricular puncture and stored in heparinized tubes on ice until analysis by ELISA as described above. Tissues were immediately placed in RPMI-1640 supplemented with 10% FBS (Avantor) and 1% pen/strep (VWR) on ice during the procedures. Following dissections, tissues were rinsed with PBS, cut using surgical scissors, and ground until paste-like in tissue grinding tubes (VWR). After mechanical digestion, enzyme digestion mix (5% FBS, 2 mg/mL Collagenase A, and 28 U/mL DNase I) was added to each tube and shaken at 37 °C for 45 minutes. The digested tissue was passed through a cell strainer with a mesh size of 100 μm, which was then rinsed with complete media (DMEM with 5% FBS and 1% pen/strep). The filtrate was collected and subsequently passed through a cell strainer with a mesh size of 40 μm. The cell-containing filtrate was centrifuged at 400×g for 5 minutes, the supernatant was aspirated, and the cell pellet was resuspended in RBC lysis buffer and incubated at room temperature for 5 minutes. PBS was added to RBC lysis buffer solution to quench the reaction. Cells were then centrifuged at 400×g for 5 min at 4 °C and supernatant was discarded. Cells were rinsed 2× with FACS buffer before being fixed using 3.7% formaldehyde for 15 minutes. Formaldehyde was removed and cells were stored in FACS buffer until fluorescent antibody staining. Cells were assessed for polarization by flow cytometry, as described below.

Flow Cytometry: Flow cytometry was used to assess macrophage polarization following in vitro and in vivo experiments. For in vitro experiments, media was removed from macrophages and cells were lifted using Accutase in DPBS (Innovative Cell Technologies). Cells were centrifuged at 300×g for 5 minutes in 1.5 mL microcentrifuge tubes (Thomas Scientific). The Accutase was aspirated and cells were rinsed 2× with sterile FACS buffer (Rockland) before being plated into 96-well FACS plates (Avantor). Cells were stained for CD14 (BV650), CD11c (PE-Cy7), CD206 (PE), CD209 (BV421), and HLA-DR (AlexaFluor 488) using fluorophore tagged primary antibodies (BD Biosciences) for 45 minutes. For in vivo experiments, cells were stained for F4/80 (BV650), CD11c (PE-Cy7), CD209 (BV421), and CD86 (AlexaFluor 700) using fluorophore tagged primary antibodies (BD Biosciences) for 45 minutes. Following staining, cells were centrifuged at 500×g for 5 minutes, supernatant was removed, and cells were washed twice in FACS buffer to remove unbound antibodies. Stained cells were analyzed on a SA3800 Spectral Analyzer (Sony) and data was analyzed using FlowJo software (BD Biosciences). Statistical analysis was performed using GraphPad. One-way ANOVA with posthoc Tukey *p<0.05, **p<0.01, ***p<0.001, ****p<0.0001.

Luminex Assay: A Luminex assay was used to measure protein levels in the serum of treated mice. Mice were euthanized via CO2 asphyxiation followed by cervical dislocation. Whole blood was collected by ventricular puncture. Serum was separated from the whole blood by spinning the whole blood at 1000×g for 10 minutes. Serum was stored at −80 °C until assay was performed. Serum samples were sent to the Human Immunology Core at The University of Pennsylvania for processing. Samples were prepared according the to the protocol provided by the manufacturer. Briefly, samples were diluted 2-fold or 20-fold in assay diluent before beginning the assay. The assay was performed on a FLEXMAP 3D luminex instrument by the staff of the human immunology core using their internal protocol. Statistical analysis was performed using GraphPad. One-way ANOVA with posthoc Tukey *p<0.05.

### REFERENCES

1 Young, R. E. et al. Systematic development of ionizable lipid nanoparticles for placental mRNA delivery using a design of experiments approach. Bioact Mater 34, 125-137, doi: 10.1016/j.bioactmat.2023.11.014 (2024).
2 Melamed, J. R. et al. Lipid nanoparticle chemistry determines how nucleoside base modifications alter mRNA delivery. J Control Release 341, 206-214, doi: 10.1016/j.jconrel.2021.11.022 (2022).
3 Evers, M. J. W. et al. State-of-the-Art Design and Rapid-Mixing Production Techniques of Lipid Nanoparticles for Nucleic Acid Delivery. Small Methods 2, 1700375, doi: https://doi.org/10.1002/smtd201700375 (2018).
4 Wice, B., Menton, D., Geuze, H. & Schwartz, A. L. Modulators of cyclic AMP metabolism induce syncytiotrophoblast formation in vitro. Exp Cell Res 186, 306-316, doi: 10.1016/0014-4827 (90) 90310-7 (1990).
5 Weber, M. et al. Cytogenomics of six human trophoblastic cell lines. Placenta 103, 72-75, doi: 10.1016/j.placenta.2020.10.011 (2021).
6 Mahon, O. R. et al. Nano-particle mediated M2 macrophage polarization enhances bone formation and MSC osteogenesis in an IL-10 dependent manner. Biomaterials 239, 119833, doi: 10.1016/j.biomaterials.2020.119833 (2020).
7 Li, M. et al. Tumor-derived exosomes deliver the tumor suppressor miR-3591-3p to induce M2 macrophage polarization and promote glioma progression. Oncogene 41, 4618-4632, doi: 10.1038/s41388-022-02457-w (2022).
8 Jenkins, N., Parekh, R. B. & James, D. C. Getting the glycosylation right: implications for the biotechnology industry. Nat Biotechnol 14, 975-981, doi: 10.1038/nbt0896-975 (1996).
9 Thomas, S. et al. Design of Glycoengineered IL-4 Antagonists Employing Chemical and Biosynthetic Glycosylation. ACS Omega 8, 24841-24852, doi: 10.1021/acsomega.3c00726 (2023).
10 Baiersdörfer, M. et al. A Facile Method for the Removal of dsRNA Contaminant from In Vitro-Transcribed mRNA. Mol Ther Nucleic Acids 15, 26-35, doi: 10.1016/j.omtn.2019.02.018 (2019).

What is claimed is:

1. An ionizable lipid nanoparticle (LNP) composition comprising C12-200 ionizable cationic lipid, 1,2-dioleoyl-sn-glycero-3-phosphoethanolamine (DOPE), cholesterol, and (1,2-dimyristoyl-sn-glycero-3-phosphoethanolamine-N-[methoxy(polyethylene glycol)-2000] (ammonium salt)) (DMPE-PEG).

2. The LNP composition of claim 1, wherein the LNP composition comprises about 25% to about 45% C12-200, about 10% to about 22% 1,2-dioleoyl-sn-glycero-3-phosphoethanolamine (DOPE), about 45% to about 55% cholesterol, and about 1.5% to about 3.5% (1,2-dimyristoyl-sn-glycero-3-phosphoethanolamine-N-[methoxy(polyethylene glycol)-2000] (ammonium salt)) (DMPE-PEG).

3. The LNP composition of claim 1, wherein the LNP composition comprises about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG).

4. The LNP composition of claim 1, wherein the LNP composition is complexed with an RNA.

5. The LNP composition of claim 4, wherein the RNA is mRNA.

6. The LNP composition of claim 5, wherein the mRNA is placental growth factor (PIGF).

7. The LNP composition of claim 5, wherein the mRNA is Interleukin-4 (IL4) or Interleukin-13 (IL13) mRNA.

8. The LNP composition of claim 5, wherein the mRNA:ionizable lipid weight ratio is about 1:2 to about 1:30.

9. The LNP composition of claim 5, wherein the mRNA:ionizable lipid weight ratio is about 1:10.

10. A method for treating a placenta-related disorder comprising administering the LNP composition of claim 6 to a pregnant subject suffering from said placenta-related disorder.

11. The method of claim 10, wherein administering is intravenous.

12. The method of claim 10, wherein the LNP composition comprises about 25% to about 45% C12-200, about 10% to about 22% 1,2-dioleoyl-sn-glycero-3-phosphoethanolamine (DOPE), about 45% to about 55% cholesterol, and about 1.5% to about 3.5% (1,2-dimyristoyl-sn-glycero-3-phosphoethanolamine-N-[methoxy(polyethylene glycol)-2000] (ammonium salt)) (DMPE-PEG).

13. The method of claim 10, wherein the composition comprises about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG).

14. The method of claim 10, wherein the placental-related disorder is preeclampsia

15. A method for treating a placenta-related disorder comprising administering the composition of claim 7 to a pregnant subject in need thereof.

16. The method of claim 15, wherein the placental-related disorder is preeclampsia.

17. The method of claim 15, wherein the LNP composition comprises about 25% to about 45% C12-200, about 10% to about 22% 1,2-dioleoyl-sn-glycero-3-phosphoethanolamine (DOPE), about 45% to about 55% cholesterol, and about 1.5% to about 3.5% (1,2-dimyristoyl-sn-glycero-3-phosphoethanolamine-N-[methoxy(polyethylene glycol)-2000] (ammonium salt)) (DMPE-PEG).

18. The method of claim 15, wherein the composition comprises about 35% C12-200, about 10% 1,2-dioleoyl-sn-glycero-3-phosphoethanolamine (DOPE), about 53.5% cholesterol, and about 1.5% (1,2-dimyristoyl-sn-glycero-3-phosphoethanolamine-N-[methoxy(polyethylene glycol)-2000] (ammonium salt)) (DMPE-PEG).

19. The method of claim 15, wherein administering is intravenous.

20. A method of delivering an mRNA to a placental tissue comprising administering the LNP composition of claim 1 complexed with the mRNA to a pregnant subject, wherein the mRNA is delivered to the placental tissue.


SEQUENCE LISTING (3 sequences)

SEQ ID NO: 1 - RNA, 1740 nt, Homo sapiens
  source 1..1740: mol_type = mRNA
agaagggagctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaa

SEQ ID NO: 2 - DNA, 4589 bp, Homo sapiens
  source 1..4589: mol_type = other DNA
acgcgcgggggcgcccctgcccaccgctcccggcagggcttttggtggccatgggggataaggggcgttgactcacccgggcggggctccgggagttgcacagaccaaggtagttccccgctccttcccccatcacggagaccctgtgggagatgccgtgggccctctactacagattaggaaacaggcccgtagaggggtcacacggccaagtagcggcactccaggcactgggggccctcgaggggaaggggcagacttctgggagtcagagccagcagctgggctgggaagcttcgagtgtggacagagagggtgggaatgacgttccctgtgggaagagagggtgggcaagcctgggatgcctctgagcgggaatccagcatgccttgtgaggagggtcacaagcacacccttgtgaggaggttgagccccatcgaggacaggacggagggagcctgagcaggcagagagggggcctggggaggcgctggttcggggaggaagtgggtaggggagaaatcttgacatcaacacccaacaggcaaatgccgtggcctctgctgtgggggtttctggaggacttctaggaaaacgagggaagagcaggaaaaggcgacatggctgcaggggccaagcccaggagccgccctccacagcactcattctgcagaagggaaatttgaggcccccagacggcaggggttgatcctgcagagactggtgagcaaaggggatcaccccaagccccagtggcactaggaacacttacaatctctgacctggactaaggctgccagcctggcccagttaagagtttcccagaaggatggcccatacactttaaattaaaggggccagacacgtgcacactacttccagccactctggaagctgaggtggggggatcgcttgagtctgggagttggaggccagcctaggcaggcaacatagtgagaccccatctccaaaaaaacaaaacaaaacaaaacaaaaaaacaccaaaaaagctcccagaaagacctctgaatctttctggatctctcagtggagacctggaaatctgaactttgacaatccctctcacagtggggccaaggaggaattaggcaagccaaaagaagtgaactttactcttctattgcctgtttgaattttgtatccaagcaagtgttacttaagtaatttaagagactggttcatcgaaaaaataaaactccccaaattcccatagctggtagactgtggtcacagccacagtgcactaagactatctgctcagcacttctggtgacccaaaagggtctgaggacaggagctcagagttgggtcagctgtccaggtactcagggttgtcacaggcaaaactgctggaactcagggcagcattgcaaatgccacgccgctctcagggccccttgcctgccgctggaattaaacccacccagatcttggaaactctgccctggacccttctcaataagtccatgagaaatcaaactctttcctttatgcgacactggattttccacaaagtaaaatcaagatgagtaaagatgtggtttctagatagtgcctgaaaaagcagagaccatggtgtcaggcgtcaccacttgggcctataaaagctgccacaagacgccaaggccacaagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggtgagtgtcggctagccagggtcctagctatgagggctccagggtgggtgattcccaagatgaggtcatgagcaggctgggcctggtcctaagatgcctgtaggtcaggaaaaatctccatggaccaaggcccggcccagccatgagggagagaggagctgggctggggggctcagcactgtggatggacctatggaggtgtctggcagactccccagggactacctgctctcctggcctggccttgtctgccactgccagctcctactcagccattcctgaacagaggacagcagagaagggccagcaccctcccagaaccatgtggcatttgccaactggattttgaccataacaatgcagccattctccccagcaccatcataggcccgcccttacaggaggattccttagtagagtccgctccttgccccactagtaacagctcacatgtctgagcactgcttacaccaggcctggtgcacgtgctttatgtgtcatttcatcactgccagccacctcaagaggcaggtacgatgaacccattctgctaaggttcagtgaggttaagtgacagaggctggattcaagccaggcctggccaacaccagagtgtccatgctcctaactgcagtgttccctcaccatcagaaggcagggcatttaatacaccagatccccaccgcctcccatctgatttgtcttggtcaacagtggcccaggccactcctacttcactcgtccccaccctggcccttcccgcaggcccctgtcctcctgccctgactatggcaagccttgcatgcagcttgtcccttactagtggtgtcaatttttttctctcagctccaagaccctaaacagtgggacctcacccctatgcctgctgttcaaagcagaaaacgaagctcaggaatgctgaggggctgccaggcctgcctctgtgccacaccagggatgcttgtggggcctgtgctggggcagacctggcctgggctgccagggcaggcccacaacccctgccagcactctgctcactgtcactttgctcccacaggctccgctctgcaatggcagcatggtatggagcatcaacctgacagctggcatggtaaggacctttgggtgcagggaggatggggcagaggctccaggccttgggcttatcttctctgagcctcccttccatggctggggttccaagcaagcttcaagtgctctcctccctcccgccataatctggccccttcccgcccaccacccagactcacctgcgccaggcatctcagccccatcttcctgcagactcacaaaaggcagctgcccaagcagggcctgacccctcggtgtcccctccccacagtactgtgcagccctggaatccctgatcaacgtgtcaggctgcagtgccatcgagaagacccagaggatgctgagcggattctgcccgcacaaggtctcagctggggtaaggcatcccccaccctctcacacccaccctgcaccccctcctgccaaccctgggctcgctgaagggaagctggctgaatatccatggtgtgtgtccacccaggggtggggccattgtggcagcagggacgtggccttcgggatttacaggatctgggctcaagggctcctaactcctacctgggcctcaatttccacatctgtacagtagaggtactaacagtacccacctcatggggacttccgtgaggactgaatgagacagtccctggaaagcccctggtttgtgcgagtcgtcccggcctctggcgttctactcatgtgctgacctctttgtcctgcagcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagata

SEQ ID NO: 3 - DNA, 8690 bp, Homo sapiens
  source 1..8690: mol_type = other DNA
atcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaaggtgagtacctatctggcaccatctctccagatgttctggtgatgctctcagtatttctaggcatgaaaacgttaacagctgctagagaagttggaactggtggttggtggcagtccagggcacacagcgaggcttctcccctgccactcttttttctgagggtttgtaggaagtttcctcagttggagggagtgagagctgctcatcaaggacttctctgtccggttggaggttaactctgtctcttgctctctcatttctgcctggaccaagactctgtgcaccgagttgaccgtaacagacatctttgctgcctccaaggtaagaagccgtcccacggtctgttttagcaaatggggagatccatccccaaatgtctgaacaagaaacttgtctaatggaaaacgagcgggcccaaattaactctaaggtgttagatgttttcaaagaacgagaagtctgatctttactcttaagcatgttttggtctttctggtttcacttgatttagaagacatgtaatagaaagcttacatgctgtagtcctgactcagatcctggtcaaagaaaagccctcttgggttttacttagctttggcatagtgcctggaacgtaggaggcactcaataaatgcctgttgaatgagagaatttttctggcccatacatttctgaaaaaccaaatactctcacagaaacagatattgagatgacaggttgagggagctttcattttgtctaagagacttcctatggcaacagaaaaggtatcgccagagcccctcctcttccacagcctggccacctaacagccctctgggttccggggctggccgtccagagctcctcagcttgctctggccggccgaactcccctccagctcggtctggaaccatcctgctgggcagcgtccagcacatccctgcttcgggctgcctgggcacctcgcctctctgcctcctgtgctgcctcacccccacccctctatctgtagtgggaggagatagatttgacagctgatagtgcattttctctgacaaacacatgactacagccgtatcaatagttttgtgcatttcagttcctgttttcatggaaacacacggctgagaatgaaagccccaaagcctcaatttcacagtggtctcctaactacctgctttccatgcaaactagggagatgatatggccaggagtgaagccctgtgtgttgggcagggtcacactccagcacccagaccatagaacagggcccatcctgcttcatgagggaaactgctcttcgggcctttagctggactatctcatttcattagttatcccgggagtccgatacaggatgagattctgaagggcaaatacacacttttttttttttttgagatagggtcttgttctgtcacccaggctggagtgcagtggtgcgatttcagctcatagcagcctccacctcccaggctcaagctatcttcctacctcagcctcccaagtagccgggacgacaggtgtgcaccaccacgcctggctaatttttgtatttttttgtagagatggagtcttgccattttgcccaggcttgtctcgaacttctgggctcaagcaatccgtccacctcggcctctcaaagtgctgggattagccactgcacctgggcaacagtttatgtgtgtgtgtgtgtgtgtgtgtgtgtgtatatatgtgtgtgtgtatatatatgtgtgtatgtatatatgtgtatgtatatgtgtgtgtgtgtgtgtgtgtgtgtgtataaaatctccaagtccatccaaccgagatggctcctactagaagccaagagtccaccgggttgagcactgggtctctggaggcctgtggcactgctgagaaggctctaacaaagccaagggaagggccacctcactagaagccaggcctggaggaagggtgagggctgagggcctggaggtaagactgcctgtggttttagacccagctctgccactgactagctgtgtggctggccttcagcacatcttcacacctctctgcacctcagtttccacatgtgaagatatgaaagtgattctgaaggtgattgcaaggttgattggaatccagctcttgagttagtgcaaagtgttattgtgagatgatataaccacgattaaaagcaagaacaggtgcagagaagcgatgattctaagaaggaggggaccgggttggaaaggatcaaaccatccaggatgccgagtctggggcaatccatctgggctgtttctggaagacccccgggtgcaggccaggacactgctgccctcccgtccttaactcccctcttcactcagtcctcactcacctccctctcacacacacaaacatctcctagaataatccccactgcctgccttcactcttacccgtctcatttgcctcccctgaacttcatcctcctggagttcacgatctcactcttcactcttttcttcccctcgaagattcagcactgcttacttacatgttaagatatttcagaacagtgaaatgttgctattttcaaaaacctacaaaggtggtatgcagaggaaaaggtacttctttgtgttcccaaagaaaacatctttccaaaatccagcctattgattttatttcttcgggggaacaagaattttagtatctctaagttgggtagcattctactcttggcagttgctggaaagaaggcactggtctaggtcctgggcttcacaggtaacacctgtcagggtgtctatgaagtcaaggctgtctgaggaacagcaaagtgggaagaagcaagctggctggctgatgaagggtttcttgggtggacaagtagttggagctatttcctatttaccaaagagagctaaagttcataattctacagagagttccataatgaacctcaaatacctctgttttttgaaggagtttctcatatacagcactagctgactatcctgggcaggatgggagataatgaatgcagtgccaatcgggctggatttatatggtcctcagtgaggctggtcaagaaccgagttagaactctcacagagtcactgccacagaagaaatctcccaagtggctgtttcctgacattcccgggagggacaggcctccttctgagtcactccctaagcagttctgaactgtgaggtcagccaggctgtccaagtgcactccctgagccactggcagacacactcagcagccagagctagacaggcaggtggtaggagtccagggccacggcagggatggagtgtcgccccctcgctgcgataccagagcaactaaaacgttaaggccttgcactaaagctgcccttaggatgcattcttttaaagtttttccatttaatgcagactcttttcaattcttattttatccttgtttcctttagaaagtcctttcaaaaatatctttagagggttttttcctatactatgtggccatatacgggtcaaaattaagtttaatttccaggctccaagccagcgtttcagaaaaatctcaccaaggtttgtggtaaaagaagcaaagggctgactttttggttttcttgaatctcactgttccctctgcagcagcatgcatgtctgcccacctccagacacacaggcaccatctgccgccccccatcagcccgtgtcccttccacctcgactcgcctacaaagcccagagaggtctgtttcttggcccccagagcccaaagatactgacacactcttacatttccaactagaatcaggaacgaggagtgactctcagtcagttcattaagtaaatgtctttctaaccgctctgcccatgggacatcacgccccacaggggaaaggggaagcttctgtagcctgggattctggtgcctcagtctgggtctagactttcctgaaaaaacgttaaaatatgaactgcattcctagaatttagcctacataaataagagatgaacacaaagatttctatagtttactcactgccgcttatttacagaagcaaaaatctgccacgataggggcctgacaaatgacagtaccactgtgcaatgcgtttctacgcagctctcaatcccatgttctctaataccaccgaagggcttaggaaatgcttatggtatatgtaaagagtaaagaagttacaaaacagtatcaacagttgacccctattttaaaaagtatttttaaaagtgtgacgatatttaccaaaatattaacagcaatagttacctctggctggtgggatgagtgaatgtatttttgttgaatatatgttacctttatagtaaatatatgttatcttgatcatcagaaaaaaaaatatgtaagaacttgaaagctgcttggacagcgctgctgatagaaacccctgagcatcttgtcactgttcttctgattcagagggtctgggtggggcaggggtggtctgagattctgtatttctaagaagctcccagtgatgtccatgctgctggtccatggaccacactttgagtatcaagggaccagagcatgtcgggggagaggctggggatagctttctttatctgaactggataaaggaactgggctcaagctaagaaccctctccaggttctgcatctttgttcttcagtgaaaaatgagaggacacaccaggccaggttcagactgagacacaatccctctcctgggttcccaatgacttgtctcttgtccattcccttctctaaggctaagggcccccaggaagagccatgtggccagaccctcacagttgctggcattccaaggagattctcactccgcatcatttggggccaaaaggccccttacagaagctctgcccaaggctcagatcaatggcacctgctcccagagcctcctctgatctcccaggacacctttccctgatctgtgcacttatctcttgctgcctggcaaaatgtcttagctcctcacttgggccatgtgctgctctcctctcccatggggagagccacacggagagtgctggccaaagcagcagagttcaggccaaaggatgtgcactcatttattcaacaggcatgcaggatttccagggaaagctggattttaaaacctctgggaacaagagcagaacctgactgagagctcatgtgggcacttttcatagcagaatagctcatgaggtatagagacacggacgcagaacgtgggctgtagcgacagatggtcctgcattctagtccccactgtgccttttcctcatgggatgactttattcaggtaccctttcggcaaaatcctccaagagaaaggaaactgggaggttctggggagaaggctgctgcgtttgcaattgggagaggttgttgacagaggtttatgtctgtggcaagcagccttccttcagtggaatacttgaagacaggtctgtagttgagcaaactcacctccatttgtcctcctggaaagaagaaatcaagaggaaaaatctctctcccatcctccaaatggagctggcacattgctatctgtggcatttgtctttccagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttggtaagctgcactgtattcctggcaagccggccgcgtggctcctggtggacagcagcctcacttctaaacactccttaggagctgcagcacccttggtcaacccattcattcattcactcattcaataagtatttgctgaagttccacaagtgctgggtgtggttctaggtgctgaggacgtgtcactaaagacacgcaggccgagtccctgttctcatggaatgttctaatgggagagttagaaaaacaaacatgtaaaatgatggccagcagtgatacgtgctacaaagaaaaacatagaaataaagaacataagagtcatgggggagggggctgacttaggagctggtgacattatctgagcagatatttgaattgagggagcaggccacatgactaactagggagaccattccaggcagaaggaggaggtatgcaaaggccttaggatggaaatgaactaacttcctgtatttaaagaccagtaggaaggccagtgtggctggatcagagtgagtgaggggtagtttccaggacagcagatcacacaaggcctttagattccaccacgagtatggcagggaacacctgcagagctttgggcaggacaaagactgtacaatctgatttacgtgatttaaaagggtcagtctggctactgtgtggtaaataggctgaaagggggaaagcatagaagcaagatggcctgttgggaggctaccacagtaaaccaggctagagatgatggtggcgtggacagaatgaagcaagatggcctgttgggaggctaccacagtaaaccaggctagagacgatggtggcgtggacagaatgaagcaagatggcctgttgggaggctaccacagtaaaccaggctagagatgatggtggcgtggacagaatgggagcagttgaggtgaacagatttgggatatgactaaaaataaaaccagaaggatttgctgacagatcggttgtagggggtaagatacaggggaggaaaagatgacctctttgttcctgcccaaacccctctggcgatggtcagtactgtttacagagagatgaaagactggcggcaaggcagggctggaggttcagcagaagatcaagagttcaattttgtacatcgtacatgtaaggtggctcttggatagccaagtgaaggtgttgagaagatggttagaaaagtctggaacttaggggagaggtcagaacttgcaatacaaaaaggagagtccttagatagatactgctgaaaatctgaatgacagaaagggagagatcaaaggactgagcctgagatcaacacatggaggtcaggagaggaggatccagccaaggggcctgaggaggagtgaccagtgaggcaggagaacactggagagtgggcggtaccccaggaagccgttgaggacactcaaggagggagggttgactgtgtcaaatgtactgaaaggacaggtcaggtgaggaccaagaaaggcccctgggtttggctgatggaggccatgggtgaggctgatgtaaatggagaggcaggaaggaaagcccagctggagtgggctcaccgaggatagggtggcgagaggagacaaagaaggaacagtgagggcagaacactctttgaagatgtttagctataaggctgcagagaaactgacccacagctgcagggtggttatggagtgagggaagctcttttaaggttgggggtatacccagcatgttaatgcacctgggggaatggtccagtggagcaggaagaactgaagagagcagaaagaggaagaatcattagggggcagaagtccttgtagcccagagtggatgttatctaatatcgagtggaggaattaattggctttagaggagaacaaggacatgtatcccctctctgggcctatcaccttgtagacaatgggataggtcatgggataggaacttggcacaacacatgttctctcttttaattctctccattatcttatgaagcaggcaagtaggcaaacaattgtcccaactttacaaaagaaactgaagcttttataaattaagtagtacatcctaagcaatacaattaataaatggtagagctgagattcaaactgaagcagtggcctgggggtagcatctggaatccttcccacctttagggctgctgtgctgcggtgctgctgtttaatggcacaggagggccacatgactgaatctctctcagcagtccaggcagtcatgcagaaggcccagtagagcaccgggcaggtctgagccagcatcttcaagttccaccctctgagcaagcacctagctgtgacacacctctccagagactgcactcccccccgcgccacccaccccaaaagcagataggtaatggtatacagtaaccatttctagaagtgtaagtagtatgcacccaaaataggcaaaacctgctggcctagtgatagagacaactcccagtcaggctagactggaggccttggttttataagtgttcaggtgacaagtgccacagtaggcttgatcaagtagacaggcaggcaagacaaatgcttaccaatgcaagctaatgaaatgtttcttttgcagaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaa